(19) United States
(12) Patent Application Publication
(10) Pub. No.: US 2023/0233403 A1
(43) Pub. Date: Jul. 27, 2023

(54) MOVING DISTANCE CALCULATION METHOD

(71) Applicant: REAL DESIGN CO.,LTD, Sendai-shi (JP)

(72) Inventors: Kenji NUNOKAWA, Sendai-shi (JP); Toshiaki TANIGUCHI, Sendai-shi (JP); Katsuhisa ABE, Sendai-sh (JP); Kenichi NOBE, Sendai-sh (JP); Jun NUNOKAWA, Sendai-sh (JP)

(73) Assignee: REAL DESIGN CO.,LTD, Sendai-shi (JP)

(21) Appl. No.: 18/000,993

(22) PCT Filed: Jun. 3, 2021
(86) PCT No.: PCT/JP2021/021265

§ 371 Date: Dec. 7, 2022

(30) Foreign Application Priority Data
Jun. 9, 2020 (JP) 2020-100350

(51) Int. Cl. A61H 31/00; A61B 5/00

(52) U.S. Cl. CPC A61H 31/005; A61B 5/0053; G06F 3/0346

(57) ABSTRACT

To calculate a moving distance with high accuracy based on an acceleration acquired from an acceleration sensor.

A mobile information terminal 1 performs gravity correction processing for an acceleration acquired from an acceleration sensor 18 to remove a gravitational acceleration component, calculates a distance by performing integration processing twice, and calculates a moving distance from extracted start and end points as compression depth. The mobile information terminal 1 notifies a rescuer who is performing cardiac massage of the compression depth in the immediately preceding compression operation so as to allow him or her to perform the cardiac massage with an adequate compression depth.

### TECHNICAL FIELD

The present invention relates to a moving distance calculation method, which is suitably applied to a case where, for example, chest compression which is one of the procedures for CRP (Cardiopulmonary Resuscitation) is performed. More specifically, the method is suitably applied to when an information processing device such as a smartphone is used to allow a rescuer to recognize appropriate compression depth during the chest compression.

### BACKGROUND ART

Non-Patent Document 1 has reported about cardiopulmonary resuscitation. It describes that the one-month survival rate of a patient in cardiopulmonary arrest abruptly decreases in a case where cardiopulmonary resuscitation is not started within 10 minutes after cardiopulmonary arrest, and that when chest compression is performed by one rescuer, he or she reaches his/her physical limit in five to six minutes.

As described above, it is widely recognized that the survival rate of a patient in cardiopulmonary arrest significantly depends on how early the treatment is started and how appropriate the treatment is. For a patient in cardiopulmonary arrest, it is very important to perform cardiac massage to repeatedly compress the patient’s chest with reliability and without delay so as to circulate the blood until AED (Automated External Defibrillator) is applied. It is said that the survival rate of the patient depends on how appropriate the cardiac massage is performed.

For example, Non-Patent Document 2 describes that when the cardiac massage is performed for an adult patient in cardiopulmonary arrest, compression depth (moving distance of body surface upon chest compression) should be set in a range of 5 cm to 6 cm, and chest compression should be performed at a rate of 100 to 200 compressions per minute. However, in most cases, non-medical personnel do not have knowledge for performing the cardiac massage for cardiopulmonary resuscitation.

As a medical device that a rescuer who performs cardiopulmonary resuscitation can use, a CPR meter for notifying the rescuer of chest compression depth and chest compression frequency is available. However, the CPR meter is allowed to be used only by medical workers or persons trained for this device. Further, the CRP meter is expensive, so that deficiency in installation thereof is supplemented by an AED. Thus, the CPR meters are installed in limited places, and thus it is difficult for ordinary persons to use the CPR meter.

On the other hand, information processing devices such as smartphones which are widely diffused are designed in a size to fit into the palm of a user, and many of them have a function of performing various computations by executing application programs installed therein and incorporate an acceleration sensor. So, there is proposed a method of using such a smartphone as an assisting device for cardiac massage (for example, see Patent Document 1).

In the method disclosed in Patent Document 1, cardiac massage is performed in a state where a smartphone executing a predetermined application program is put between the chest of a patient and the hand of a rescuer. At this time, the smartphone performs computation such as integration based on an acceleration value acquired from an acceleration sensor to calculate a moving distance, calculates a period from the interval between appearing peaks, and notifies the rescuer of information concerning the moving distance and period. Thus, the smartphone can guide a user to perform cardiac massage with adequate compression depth and with an appropriate period.

### CITATION LIST

### Patent Document

Patent Document 1: Korean Registered Patent No. 10-1,054,722

### Non-Patent Document

Non-Patent Document 1: Tokyo Fire Department “Reports on Reforms Addressing Visualization <Rescue Operation> overall version”, p.50 [online], Nov. 28, 2017 [searched on May 22, 2020] URL:https://wwww.tfd.metro.tokyo.lg.jp/portal/data/all-a.pdf.
NON-Patent Document 2: American Heart Association “Highlights of 2015 Guidelines Update for Cardiopulmonary Resuscitation and Emergency Cardiovascular Care”, p.7, [online], October 2015 [searched on May 22, 2020] URL:https://eccguidelines.heart.org/wp-content/uploads/2015/10 /2015-AHA-Guidelines-Highlights-Japanese.pdf>

### DISCLOSURE OF THE INVENTION

### Problems to Be Solved by the Invention

However, the acceleration sensor incorporated in the smartphone detects an acceleration including a gravitational acceleration. This may significantly deteriorate the accuracy in, e.g., moving distance to be calculated. Further, when an acceleration due to the movement caused during cardiac massage is smaller than a gravitational acceleration, no peak may appear in the moving distance, which may lead to deterioration in detection accuracy of a period.

The present invention has been made in view of the above points, and an object thereof is to propose a moving distance calculation method capable of calculating a moving distance with high accuracy based on an acceleration acquired from an acceleration sensor.

### Means for Solving the Problems

To solve the above problems, a moving distance calculation method according to the present invention includes: an acquisition step of acquiring an acceleration from an acceleration sensor during a plurality of reciprocations; a gravity correction step of performing gravity correction processing for the acceleration using a gravity correction value corresponding to a gravitational acceleration; a first integration step of calculating a velocity by performing integration processing for the acceleration that has been subjected to the gravity correction processing; a second integration step of calculating a distance by performing integration processing for the velocity; and a moving distance calculation step of extracting, from the distance, feature points corresponding to a start point and an end point in the forward stroke of the reciprocation and calculating a moving distance of the forward stroke of the reciprocation based on a difference value of the distance between the extracted start and end points.

Further, a moving distance calculation method according to another aspect of the present invention is a moving distance calculation method used in an information processing system having a first information processing device put on the arm of a user and a second information processing device communicably connected to the first information processing device and incudes: an acquisition step of acquiring an acceleration from an acceleration sensor during a plurality of reciprocations of the first information processing device; a gravity correction step of performing gravity correction processing for the acceleration using a gravity correction value corresponding to a gravitational acceleration; a first integration step of calculating a velocity by performing integration processing for the acceleration that has been subjected to the gravity correction processing; a second integration step of calculating a distance by performing integration processing for the velocity; and a moving distance calculation step of extracting, from the distance, feature points corresponding to a start point and an end point in the forward stroke of the reciprocation and calculating a moving distance of the forward stroke of the reciprocation based on a difference value of the distance between the extracted start and end points, the moving distance calculation method further including a transmission step of transmitting the acceleration, the velocity, and the distance or the moving distance from the first information processing device to the second information processing device.

According to the present invention, the gravity correction processing is performed for the acceleration to thereby remove a component corresponding to the gravity from the acceleration. Thus, it is possible to allow a change corresponding to the reciprocation to appear in the velocity obtained by integrating the acceleration and distance obtained by integrating the velocity without being buried in a component corresponding to the gravitational acceleration and thus to adequately calculate the moving distance.

### Advantageous Effects of the Invention

According to the present invention, it is possible to achieve a moving distance calculation method capable of calculating a moving distance with high accuracy based on an acceleration acquired from an acceleration sensor.

### BRIEF DESCRIPTION OF THE DRAWINGS

FIG. 1 is a schematic view illustrating the outer appearance of a mobile information terminal.

FIG. 2 is a block diagram illustrating the circuit configuration of the mobile information terminal.

FIGS. 3A to 3C are graphs illustrating various waveforms obtained without performing correction processing.

FIG. 4 is a block diagram illustrating a functional block configuration according to a first embodiment.

FIG. 5 is a flowchart illustrating a moving distance calculation processing routine according to the first embodiment.

FIGS. 6A to 6D are graphs illustrating various waveforms according to the first embodiment.

FIG. 7 is a graph illustrating a case where the moving distance cannot be calculated adequately in the first embodiment.

FIG. 8 is a block diagram illustrating a functional block configuration according to a second embodiment.

FIG. 9 is a flowchart illustrating a moving distance calculation processing routine according to the second embodiment.

FIG. 10 is a flowchart illustrating velocity tilt correction processing routine according to the second embodiment.

FIGS. 11A to 11D are graphs illustrating various waveforms (1) according to the second embodiment.

FIG. 12 is a graph illustrating various waveforms (2) according to the second embodiment.

FIG. 13 is a graph illustrating a case where the moving distance cannot be calculated adequately in the second embodiment.

FIG. 14 is a block diagram illustrating a functional block configuration according to a third embodiment.

FIG. 15 is a flowchart illustrating a moving distance calculation processing routine according to the third embodiment.

FIG. 16 is a flowchart illustrating center value correction processing routine according to the third embodiment.

FIGS. 17A to 17D are graphs illustrating various waveforms (1) according to the third embodiment.

FIGS. 18A and 18B are graphs illustrating various waveforms (2) according to the third embodiment.

FIG. 19 is a graph illustrating a case where the moving distance cannot be calculated adequately in the third embodiment.

FIG. 20 is a block diagram illustrating a functional block configuration according to a fourth embodiment.

FIG. 21 is a flowchart illustrating a moving distance calculation processing routine according to the fourth embodiment.

FIG. 22 is a flowchart illustrating distance tilt correction processing routine according to the fourth embodiment.

FIGS. 23A to 23D are graphs illustrating various waveforms (1) according to the fourth embodiment.

FIGS. 24A to 24C are graphs illustrating various waveforms (2) according to the fourth embodiment.

FIGS. 25A to 25D are graphs illustrating various waveforms (1) when compression operation is performed by a rescuer in the fourth embodiment.

FIGS. 26A to 26C are graphs illustrating various waveforms (2) when compression operation is performed by a rescuer in the fourth embodiment.

FIG. 27 is a graph illustrating a velocity waveform including a drift component.

FIG. 28 is a block diagram illustrating a functional block configuration in a preliminary velocity coefficient calculation processing according to a fifth embodiment.

FIG. 29 is a flowchart illustrating a preliminary velocity coefficient calculation processing routine RT according to the fifth embodiment.

FIG. 30 is a block diagram illustrating a functional block configuration in a moving distance calculation processing according to the fifth embodiment.

FIG. 31 is a flowchart illustrating a moving distance calculation processing routine according to the fifth embodiment.

FIG. 32 is a graph illustrating the relation between the frequency of the compression operation and distance to be calculated.

FIG. 33 is a block diagram illustrating a functional block configuration in a correction coefficient calculation processing according to a sixth embodiment.

FIG. 34 is a flowchart illustrating a correction coefficient calculation processing routine according to the sixth embodiment.

FIG. 35 is a block diagram illustrating a functional block configuration in a correction coefficient application processing according to the sixth embodiment.

FIG. 36 is a flowchart illustrating a correction coefficient application processing routine according to the sixth embodiment.

FIG. 37 is a view illustrating the entire configuration of an information processing system according to a seventh embodiment.

FIG. 38 is a block diagram illustrating the circuit configuration of an arm wearing information terminal according to the seventh embodiment.

FIG. 39 is a block diagram illustrating a functional block configuration according to the seventh embodiment.

FIG. 40 is a flowchart illustrating a moving distance calculation processing sequence according to the seventh embodiment.

FIG. 41 is a block diagram illustrating a functional block configuration according to an eighth embodiment.

FIG. 42 is a flowchart illustrating a moving distance calculation processing sequence according to the eighth embodiment.

### BEST MODE FOR CARRYING OUT THE INVENTION

Hereinafter, embodiments for practicing the present invention will be described using the drawings.

### 1. First Embodiment

### 1-1. Configuration of Mobile Information Terminal

As illustrated in FIG. 1, a mobile information terminal 1 according to a first embodiment is, for example, a smartphone, in which various components are incorporated inside a flat rectangular parallelepiped housing 2 thereof and a touch panel 3 is incorporated in the front face thereof. The mobile information terminal 1 further incorporates therein a speaker 4 emitting sound and a microphone 5 converting sound into an electrical signal.

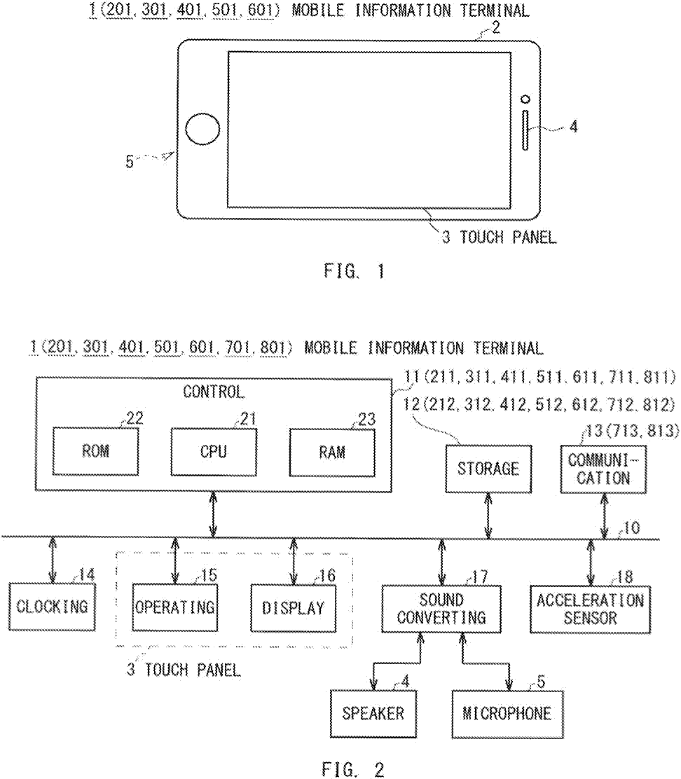

The mobile information terminal 1 is a so-called information processing device, in which, as illustrated in FIG. 2 illustrating a schematic circuit configuration, a control part 11, a storage part 12, a communication part 13, a clocking part 14, an operating part 15, a display part 16, a sound converting part 17, and an acceleration sensor 18 are connected through a bus 10.

The control part 11 has a CPU (Central Processing Unit) 21, a ROM (Read Only Memory) 22, and a RAM (Random Access Memory) 23. When a power supply is powered on, the CPU 21 reads out, from the ROM 22 and storage part 12, programs such as an operating system or various applications and executing the readout programs while using the RAM 23 as a work area. The control part 11 thus executes various processing to totally control the mobile information terminal 1.

The storage part 12 is constituted by, e.g., a flash memory and stores therein various programs and data. The communication part 13 establishes communication connection with a not illustrated base station according to a mobile communication standard such as 4G (4th generation) or 5G (5th Generation) and transmits/receives various information. The communication part 13 serves also as the interface of a wireless LAN conforming to a standard such as IEEE (Institute of Electrical and Electronics Engineers) 802.11a/b/g/n/ac and transmits/receives various information to/from a not illustrated base station (or master set). The clocking part 14 measures time.

The operating part 15 is a touch sensor constituting a part of the touch panel 3. The operating part 15 detects contact of user’s finger with the touch panel 3 and supplies information related to the contact position to the control part 11 as a user’s operation input. The display part 16 is a liquid crystal display panel constituting a part of the touch panel 3 and displays various screens including characters and graphics under the control of the control part 11. The display part 16 displays and updates screens at a frame rate of, e.g., 30 frames per second.

The sound converting part 17 performs mutual conversion processing between sound data and sound signals and various processing concerning sound data or sound signals. The sound converting part 17 is connected with the above-mentioned speaker 4 and microphone 5. For example, the sound converting part 17 converts sound data supplied from the control part 11 into sound signals and makes the speaker 4 emit sound. Further, the sound converting part 17 converts sound signals generated by collecting surrounding sound with the microphone into sound data and supplies the sound data to the control part 11.

The acceleration sensor 18 detects an acceleration and generates and outputs a detection signal with a voltage level corresponding to the magnitude of the detected acceleration. That is, the acceleration sensor 18 outputs an acceleration in the form of an analog value.

### 1-2. Calculation of Moving Distance

The following describes the basic principle and processing procedure of moving distance calculation using the mobile information terminal 1 when cardiopulmonary resuscitation (CPR) is performed. As in the case of the CRP meter or the method disclosed in Patent Document 1, the first embodiment assumes a case where a rescuer repeatedly performs compression operation with the mobile information terminal 1 put between the chest of a patient in cardiopulmonary arrest and the hand of the rescuer, that is, a case where the mobile information terminal 1 reciprocates substantially vertically.

As is widely known, velocity can be obtained by integrating an acceleration, and a moving distance can be obtained by integrating the obtained velocity. However, on the ground, a gravitational acceleration of about 1G (about 9.8 [m/s²]) is added to an acceleration sensor. Thus, an acceleration detected by the acceleration sensor includes a component due to gravity as noise with a DC component.

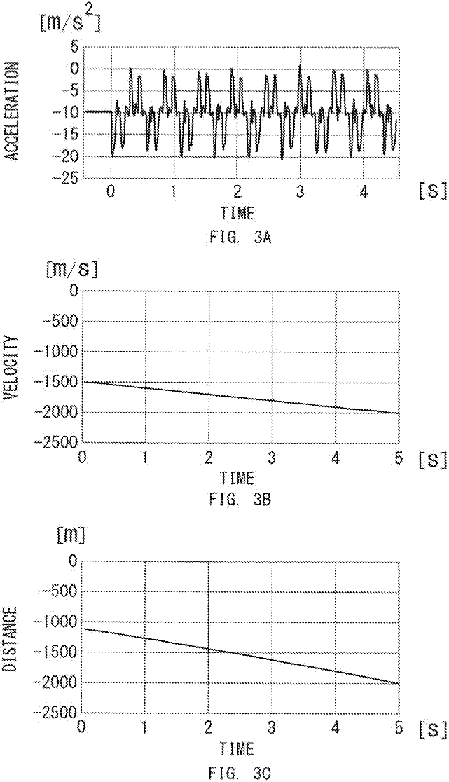

For example, when an acceleration acquired from an acceleration sensor 18 of the mobile information terminal 1 during cardiopulmonary resuscitation is shown in a graph, a waveform fluctuates centering around -1G (about -9.8 [m/s²]) (FIG. 3A). This acceleration includes a gravitational acceleration component, so that the waveform fluctuates centering not around 0 but around -1G.

The acceleration of FIG. 3A is a value obtained when a dedicated tool (not illustrated) is used to repeatedly compresses the chest of a doll prepared for training of cardiopulmonary resuscitation. Unless otherwise noted, the waveforms to be described later represent an acceleration obtained when the dedicated tool is used to repeatedly compresses the doll chest and various values obtained based on the acceleration. In the example of FIG. 3A, according to the descriptions of Non-Patent Document 2, compression operation is performed with compression distance (i.e., compression depth) set to 55 [mm] and compression frequency set to 110 per minute (110 [rpm]).

When, in the acceleration detected by the acceleration sensor 18, a noise component (gravitational acceleration and the like) is larger than a component due to vertical movement caused by the compression operation, velocity obtained by integrating the acceleration and distance obtained by integrating the velocity have the waveforms as illustrated in, e.g., FIGS. 3B and 3C, respectively. The calculated velocity and distance illustrated in FIGS. 3B and 3C each hardly exhibit a feature of periodically changing according to the vertical reciprocation in association with the compression operation. In this case, the start and end points of the forward stroke of the reciprocation cannot be detected adequately from the waveform illustrated in FIG. 3C. Further, compression depth and period are also very difficult to calculate.

To cope with this, the mobile information terminal 1 according to the first embodiment is configured to calculate a velocity value and a moving distance after removing the component due to the gravitational acceleration. For example, when the control part 11 of the mobile information terminal 1 receives an instruction to start processing corresponding to cardiopulmonary resuscitation through a user’s input operation in a state where a predetermined operating system is in operation, it reads out a predetermined cardiopulmonary resuscitation program (not illustrated) from the storage part 12 as an application program and executes it.

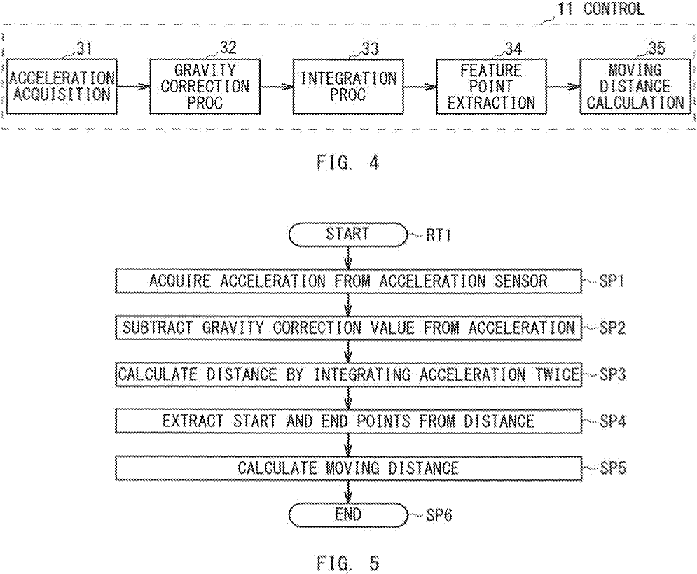

After starting the cardiopulmonary resuscitation program, the control part 11 performs predetermined initialization processing and preparation processing and then reads out and executes a moving distance calculation program as a sub routine. As illustrated in FIG. 4, the control part 11 forms therein a plurality of functional blocks including an acceleration acquisition part 31, a gravity correction processing part 32, an integration processing part 33, a feature point extraction part 34, and a moving distance calculation part 35.

The acceleration acquisition part 31 acquires an acceleration value from the acceleration sensor 18. The acceleration acquisition part 31 uses a predetermined A/D (Analog/Digital) converter (not illustrated) to convert an analog value output from the acceleration sensor 18 into a digital value to thereby acquire a digital acceleration value. The gravity correction processing part 32 corrects the acquired acceleration value to remove the gravitational acceleration component.

The integration processing part 33 performs first integration processing for the acceleration value to calculate a velocity value and then performs second integration processing to calculate distance. The feature point extraction part 34 extracts, from the waveform of the distance, a point (feature point) representing a value (feature value) (e.g., a local maximum value or a local minimum value) having a feature. The moving distance calculation part 35 calculates the moving distance in one compression operation based on the feature value.

After forming the above functional blocks, the control part 11 starts a moving distance calculation processing routine RT1 illustrated in FIG. 5 and proceeds to step SP1. In step SP1, the control part 11 makes the acceleration acquisition part 31 (FIG. 4) convert an analog acceleration value acquired from the acceleration sensor 18 into a digital acceleration value by means of a predetermined A/D converter (not illustrated) and then proceeds to step SP2.

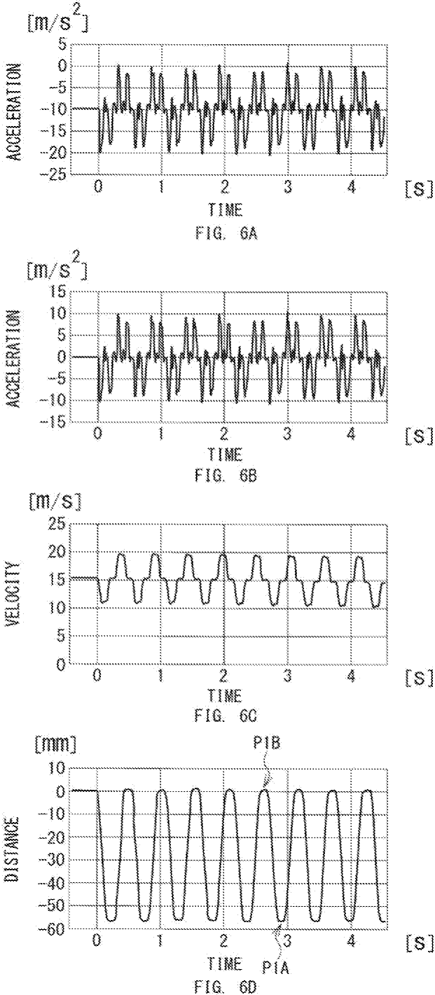

If the acceleration acquired from the acceleration sensor 18 does not include the gravitational acceleration or various noise, the acceleration exhibits a waveform fluctuating in positive and negative directions for each period of compression operation, substantially centering around 0 [m/s²] in a situation where compression operation is repeatedly performed with the mobile information terminal 1 put on the chest of a patient. Actually, however, the acceleration includes a component (drift component) such as the gravitational acceleration or various noise and thus fluctuates centering around a value deviated from 0 [m/s²], specifically, about -9.8 [m/s²] as illustrated in FIG. 6A corresponding to FIG. 3A.

At this time, the A/D converter converts the acceleration from an analog value to a digital value at a predetermined sampling frequency (e.g., 200 [Hz]). That is, the converted digital acceleration value is a value for each predetermined sampling period (e.g., 1/200 [s]).

In step SP2, the control part 11 uses the gravity correction processing part 32 (FIG. 4) to subtract a gravity correction value from the acceleration as gravity correction processing and then proceeds to step SP3. The gravity correction value is a value (9.80665 [m/s²]) of a standard gravitational acceleration and is previously stored in the storage part 12. Thus, by subtracting the gravity correction value from the acceleration, the control part 11 can remove almost completely the gravitational acceleration component included in the acceleration. As a result, the acceleration after correction exhibits a waveform fluctuating in positive and negative directions substantially centering around 0 [m/s²], as illustrated in FIG. 6B.

In step SP3, the control part 11 uses the integration processing part 33 (FIG. 4) to perform integration processing (first integration processing) for the acceleration to calculate a velocity. The velocity obtained at this time exhibits a waveform substantially periodically fluctuating between about 10 [m/s] and about 20 [m/s] as illustrated in FIG. 6C. Subsequently, the control part 11 uses the integration processing part 33 to perform integration processing (second integration processing) for the velocity to calculate a distance and then proceeds to step SP4. The distance obtained at this time exhibits a waveform substantially periodically fluctuating between about 0 [mm] and about -55 [mm] as illustrated in FIG. 6D.

In step SP4, the control part 11 uses the feature point extraction part 34 (FIG. 4) to extract the feature point representing the feature value in the waveform of the distance to identify the start and end points in the immediately preceding compression operation and then proceeds to step SP5. Specifically, the feature point extraction part 34 extracts, as the end point, a feature point appearing as the latest local minimum value, and extracts, as the start point, a feature point appearing as the local maximum value immediately before the latest local minimum value, followed by storage of the values and timings thereof in the storage part 12. After that, the control part 11 proceeds to step SP5.

At this time, the feature point extraction part 34 regards, as the local minimum point, the distance at a time point when the distance value turns from decrease to increase with lapse of time, in other words, the distance at a time point when the sign of the velocity obtained by differentiating the distance with respect to time turns from negative to positive. Further, the feature point extraction part 34 regards, as the local maximum point, the distance at a time point when the distance value turns from increase to decrease with lapse of time, in other words, the distance at a time point when the sign of the velocity obtained by differentiating the distance with respect to time turns from positive to negative.

For example, assuming that the current time is 3 [s] in FIG. 6D, the feature point extraction part 34 determines, as the end point, a point P1A which is the latest local minimum value and determines, as the start point, a point P1B which is the local maximum value immediately before the latest local minimum value.

In step SPS, the control part 11 uses the moving distance calculation part 35 (FIG. 4) to calculate a difference value between the start point and end point to thereby calculate the moving distance from the start point to end point. Thereafter, the control part 11 proceeds to step SP6 and ends the moving distance calculation processing routine RT1.

The control part 11 can calculate also a period which is a time required for one compression operation based on the time difference between the latest start point and a start point extracted immediately before the latest start point. Thereafter, the control part 11 returns to the cardiopulmonary resuscitation program and displays the obtained moving distance as compression depth on the display part 16 (FIG. 2) together with the period as notification to the rescuer.

### 1-3. Effects, Etc.

With the above configuration, the mobile information terminal 1 according to the first embodiment executes the moving distance calculation program as a sub routine of the cardiopulmonary resuscitation program to perform the gravity correction processing of subtracting the gravity correction value from the acceleration acquired from the acceleration sensor 18 to thereby remove the gravitational acceleration component. Further, the mobile information terminal 1 performs integration processing twice for the resultant acceleration to calculate the distance and extracts the start and end points to calculate the moving distance as the compression depth in the immediately preceding compression operation.

That is, the mobile information terminal 1 can calculate the moving distance with high accuracy based on the acceleration acquired from the acceleration sensor 18 incorporated therein. Then, the mobile information terminal 1 displays the moving distance on the display part 16 as the compression depth to notify the rescuer who is performing cardiac massage of the compression depth in the immediately preceding compression operation in the form of a numeric value so as to allow him or her to perform the cardiac massage with an adequate compression depth.

In other words, by using an acceleration value acquired from the acceleration sensor 18 incorporated in widely diffused smartphones, the mobile information terminal 1 can achieve the same function as CPR meters installed at limited places.

That is, the mobile information terminal 1 can assist a rescuer to perform cardiac massage with an optimum chest compression depth and an optimum period (compression frequency) as recommended in Non-Patent Document 2. Thus, when ordinary persons who do not have medical knowledge and experiences encounter a patient in cardiopulmonary arrest incidentally, the mobile information terminal 1 can function as an assisting device for cardiac massage to allow them to adequately perform cardiac massage.

Further, as illustrated in FIG. 6D, the waveform of the distance returns to about “0” for each compression operation, so that the mobile information terminal 1 can calculate the compression depth in each compression operation with high accuracy.

As a smartphone like the mobile information terminal 1, there is one that incorporates, in addition to the acceleration sensor, a sensor (e.g., an atmospheric pressure sensor) that can detect another physical quantity. Here, it can be considered that a smartphone incorporating an atmospheric pressure sensor uses atmospheric pressure detected by the atmospheric pressure sensor to remove the gravitational acceleration component included in the acceleration value acquired from the acceleration sensor. However, in this case, the atmospheric pressure sensor and accelerating sensor differ from each other in sampling frequency, thus requiring various conversion processing or various correction processing, which may lead to an increase in processing load, a deterioration in accuracy, and the like.

On the other hand, the mobile information terminal 1 according to the present embodiment performs correction, without using another sensor, by subtracting the previously stored gravity correction value (i.e., standard gravitational acceleration) from the acceleration value acquired from the acceleration sensor 18. Thus, the mobile information terminal 1 can satisfactorily remove the gravitational acceleration component by simple computation without requiring another sensor.

With the above configuration, the mobile information terminal 1 according to the first embodiment performs the gravity correction processing for the acceleration acquired from the acceleration sensor 18 to remove the gravitational acceleration component, performs integration processing twice for the resultant acceleration to calculate the distance, and calculates the moving distance from the extracted start and end points as the compression depth. Then, the mobile information terminal 1 notifies the rescuer who is performing cardiac massage of the compression depth in the immediately preceding compression operation so as to allow him or her to perform the cardiac massage with an adequate compression depth.

### 2. Second Embodiment

A mobile information terminal 201 (FIGS. 1 and 2) according to a second embodiment differs from the mobile information terminal 1 according to the first embodiment in that it has a control part 211 and a storage part 212 in place of the control part 11 and storage part 12. Other configurations are the same as those of the mobile information terminal 1 according to the first embodiment.

Like the control part 11 according to the first embodiment, the control part 211 includes a CPU 21, a ROM 22 and a RAM 23 and is configured to read out various programs from the storage part 212 and execute the readout programs. The storage part 212 stores a moving distance calculation program partly different from that of the first embodiment.

### 2-1. Calculation of Moving Distance

In the first embodiment, the mobile information terminal 1 subtracts the gravity correction value from the acceleration value acquired from the acceleration sensor 18 to remove the gravitational acceleration component. In this configuration, when the gravitational acceleration component included in the acceleration value acquired from the acceleration sensor 18 is equal to the gravity correction value, velocity and distance can be calculated with high accuracy.

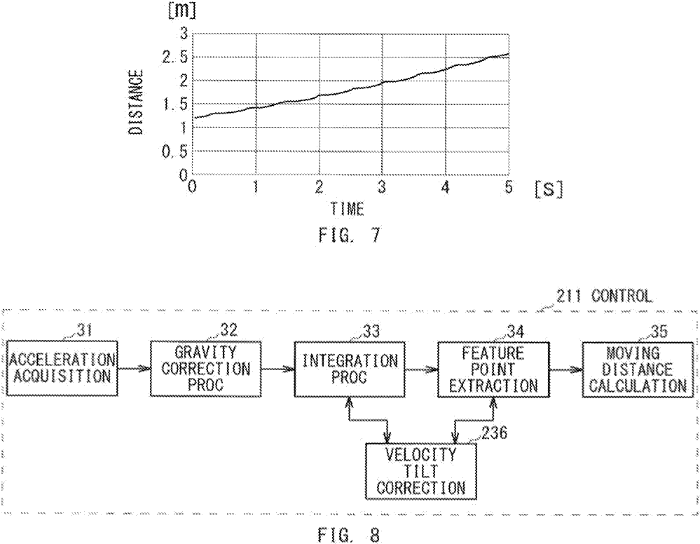

However, it is known that the gravitational acceleration value slightly varies from date to date and from location to location. Thus, only performing the gravity correction processing in the mobile information terminal 1 according to the first embodiment may fail to completely remove the gravitational acceleration component from the acceleration acquired from the acceleration sensor 18. In this case, as illustrated in FIG. 7, the local maximum and minimum values do not appear clearly in the waveform of the distance, so that the mobile information terminal 1 may fail to adequately calculate the moving distance.

To cope with this, the mobile information terminal 201 according to the second embodiment has a moving distance calculation program partly different from that of the first embodiment and is configured to perform tilt correction processing (details will be described later) with respect to the velocity in addition to the above-described gravity correction processing with respect to the acceleration.

Specifically, when reading out the moving distance calculation program from the storage part 212 and executing the program, the control part 211 of the mobile information terminal 201 forms therein a plurality of functional blocks as illustrated in FIG. 8 corresponding to FIG. 4. The control part 211 forms a velocity tilt correction part 236, in addition to the acceleration acquisition part 31, gravity correction processing part 32, integration processing part 33, feature point extraction part 34, and moving distance calculation part 35 (which are the same as those of the first embodiment). The velocity tilt correction part 236 performs correction to eliminate a velocity tilt. Specifically, when the velocity waveform has a tendency of tilting, that is, the velocity has a tendency of increasing or decreasing with lapse of time, the velocity tilt correction part 236 eliminates the velocity tilt (details will be described later).

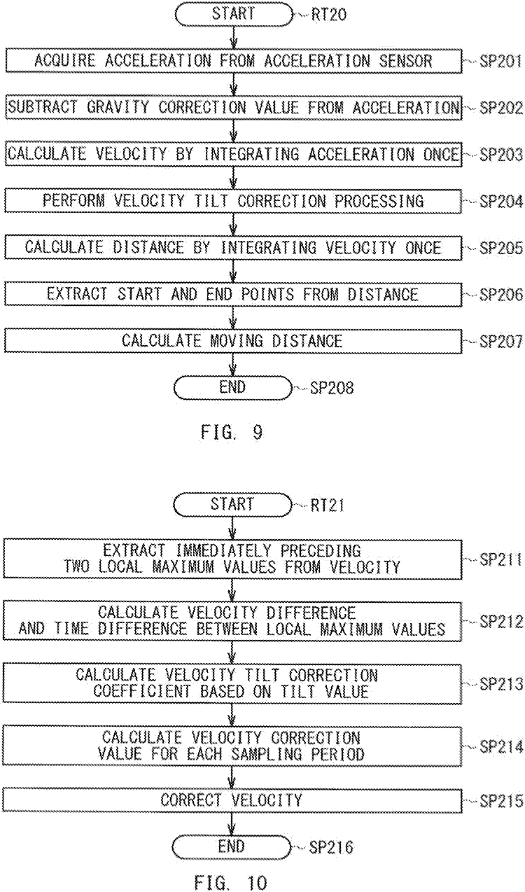

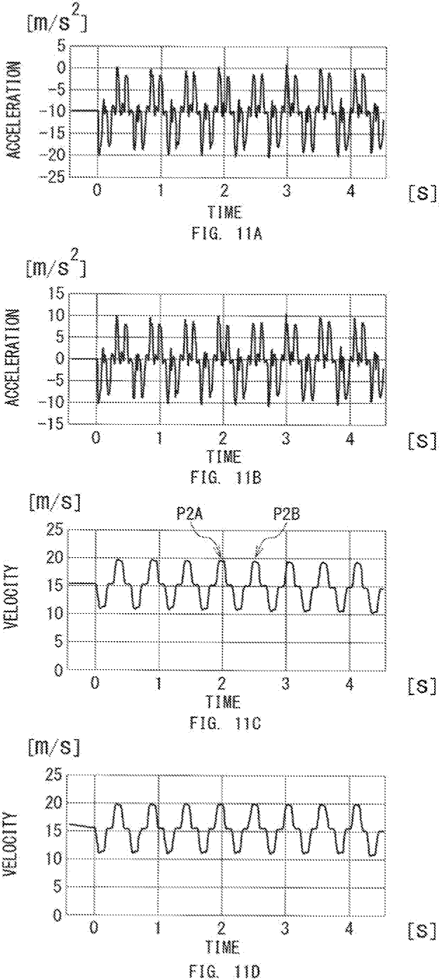

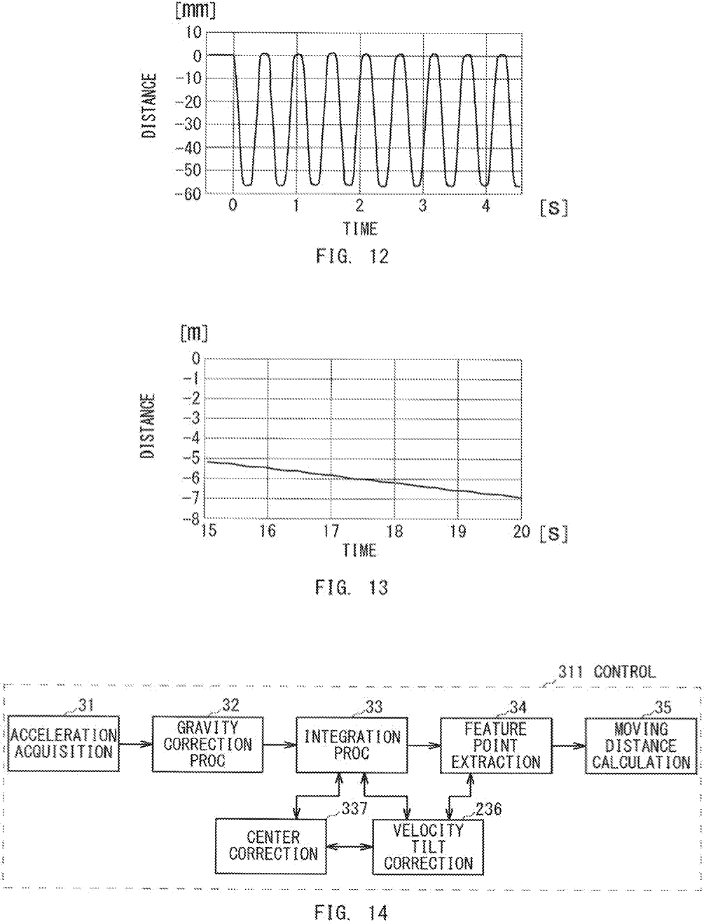

After forming the above functional blocks, the control part 11 starts a moving distance calculation processing routine RT20 illustrated in FIG. 9 corresponding to FIG. 5 and proceeds to step SP201. In steps SP201 and SP202, the control part 211 performs the same processing as those in steps SP1 and SP2 of the moving distance calculation processing routine RT1 (FIG. 5) according to the first embodiment. Through this processing, the control part 211 subtracts the gravity correction value from the acceleration whose waveform is illustrated in FIG. 11A which is the same as FIG. 6A to obtain the acceleration whose waveform is illustrated in FIG. 11B which is the same as FIG. 6B and proceeds to step SP203.

In step SP203, the control part 211 uses the integration processing part 33 (FIG. 8) to perform integration processing once for the acceleration to calculate a velocity and proceeds to step SP 204. The velocity obtained at this time has a waveform substantially periodically fluctuating between about 10 [m/s] and 20 [m/s] as illustrated in FIG. 11C which is the same as FIG. 6C. In this velocity waveform, the local maximum and minimum values appear periodically.

In step SP204, the control part 211 performs velocity tilt correction processing. A basic approach to the velocity tilt correction processing will herein be described. The acceleration based on which the velocity is acquired through the processing up to step SP203 may include a part of the drift component (component due to the gravitational acceleration or the like) that has remained without being removed by the correction processing using the gravity correction value.

In the original acceleration, this drift component is less likely to significantly vary in a relatively short time period of about 0.5 [s] corresponding to one compression operation and can thus be regarded as an approximately constant value. Thus, in terms of a sectional period sectioned by a time corresponding to one period, a velocity increasing or decreasing at a constant tilt angle in the velocity obtained by integrating the acceleration is added to the original velocity due to compression operation. In other words, a velocity represented by a linear function using time as a variable.

Thus, in the velocity tilt correction processing, focusing on two consecutive ones of the local maximum values periodically appearing in the velocity waveform and a time period therebetween, a velocity tilt correction coefficient for correcting the velocity is calculated based on a difference between velocities at the two local maximum values and a time difference between the two local maximum values, and the velocity is corrected using the calculated velocity tilt correction coefficient.

Specifically, the control part 211 starts a velocity tilt correction processing routine RT21 illustrated in FIG. 10 as a sub routine and then proceeds to SP211. In step SP211, the control part 211 uses the feature point extraction part 34 (FIG. 8) to extract, from feature points appearing in the velocity waveform (FIG. 11C), feature points representing immediately preceding two consecutive local maximum values, determines the extracted feature points as a start feature point and an end feature point in the order of appearance, and then proceeds to step SP212. For descriptive convenience, the start feature point and end feature point will be sometimes referred to also as “a velocity start feature point and a velocity end feature point”.

The feature point extraction part 34 extracts, as the local maximum value, a time point at which the velocity turns from increase to decrease in the same manner as extraction of the local maximum and minimum values of the distance in the first embodiment. Specifically, the feature point extraction part 34 determines a velocity at a time point when the sign of the acceleration obtained by differentiating the velocity turns from positive to negative as the local maximum value. For example, assuming that the current time is 3 [s] in FIG. 11C, the feature point extraction part 34 determines points P2A and P2B which are immediately preceding two consecutive local maximum values as the start feature point and end featured point, respectively, in the order of appearance.

In step SP212, the control part 211 uses the velocity tilt correction part 236 (FIG. 8) to calculate differences in velocity and time between the start feature point and end feature point as a velocity difference and a time difference, respectively and then proceeds to step SP213. In step SP213, the control part 211 uses the velocity tilt correction part 236 (FIG. 8) to subtract the velocity difference by the time difference to thereby calculate a velocity tilt correction coefficient representing the ratio of the velocity relative to the time and then proceeds to step SP214.

In step SP214, the control part 211 uses the velocity tilt correction part 236 (FIG. 8) to calculate a velocity correction value for the velocity every sampling period in between the start feature point and end feature point and then proceeds to step SP215. Specifically, the control part 211 multiplies an elapsed time from the start feature point to each sampling period by the velocity tilt correction coefficient to calculate the velocity correction value (hereinafter, referred to also as “velocity tilt correction value”) in each sampling period.

In step SP215, the control part 211 uses the velocity tilt correction part 236 (FIG. 8) to subtract the velocity correction value in each sampling period from the velocity in between the start feature point to end feature point to thereby correct the velocity. Further, at this time, the velocity tilt correction part 236 subtracts also the velocity at the start feature point from the velocity in between the start feature point to end feature point.

As a result, as illustrated in FIG. 11D, the velocities at the start feature point and end feature point have the same value after correction, and increase or decrease of the velocity due to the drift component is eliminated. Further, after correction, the velocities at the start feature point and at the end feature point both have a value of 0 [m/s].

After that, the control part 211 proceeds to step SP216 to end the velocity tilt correction processing routine RT21, then returns to step SP204 in the moving distance calculation processing routine RT20 (FIG. 9), and then proceeds to step SP205.

In step SP205, the control part 211 uses the integration processing part 33 (FIG. 8) to perform integration processing once for the velocity to thereby calculate the distance and then proceeds to step SP206. The distance obtained at this time has a waveform substantially periodically fluctuating between about 0 [mm] and -55 [mm] as illustrated in FIG. 12 corresponding to FIG. 6D.

After that, in steps SP206 and SP207, the control part 211 performs the same processing as those in steps SP4 and SP5 of the moving distance calculation processing routine RT1 (FIG. 5) according to the first embodiment. As a result, the control part 211 identifies the start and end points in the immediately preceding compression operation and calculates the distance between the start and end points. Thereafter, the control part 211 proceeds to step SP208 to end the moving distance calculation processing routine RT20.

### 2-2. Effects, Etc.

With the above configuration, the mobile information terminal 201 according to the second embodiment executes the moving distance calculation program to perform the gravity correction processing for the acceleration acquired from the acceleration sensor 18 and further the velocity tilt correction processing for the velocity obtained by integrating the resultant acceleration, followed by further integration to calculate the distance. Thereafter, as in the first embodiment, the mobile information terminal 201 extracts the start and end points from the distance to calculate the moving distance as the compression depth in the immediately preceding compression operation.

That is, the mobile information terminal 201 performs two stages of the correction processing based on the acceleration acquired from the acceleration sensor 18 incorporated therein to thereby calculate the moving distance with higher accuracy than in the first embodiment. Then, as in the first embodiment, the mobile information terminal 201 displays the moving distance on the display part 16 as the compression depth to notify the rescuer who is performing cardiac massage of the compression depth in the immediately preceding compression operation in the form of a numeric value so as to allow him or her to perform the cardiac massage with an adequate compression depth.

In particular, with the velocity tilt correction processing, the mobile information terminal 201 can sufficiently remove a part of the drift component that has not been removed by the gravity correction processing which is the same processing as in the first embodiment and thus can significantly improve the calculation accuracy of the distance as compared to the first embodiment.

Further, in the velocity tilt correction processing, the mobile information terminal 201 determines the two consecutive local maximum values appearing in the velocity waveform as the start feature point and end feature point, regards the drift component remaining in the acceleration based on which the velocity is acquired as constant in a time period of about 0.5 [s] corresponding to between the start feature point and end feature point, and treats it as one increasing or decreasing at a constant tilt angle in the velocity.

Thus, the mobile information terminal 201 can satisfactorily correct a part corresponding to the drift component while significantly reduce a processing load required for computation as compared to a case where the drift component is treated as a constantly varying value. As a result, the mobile information terminal 201 which is a smartphone can calculate, in a range within which real time computation can be achieved by the computation processing capability of the control part 211, a highly accurate distance from which the drift component has effectively been removed.

In another view point, compression operation is repeatedly performed with downward movement by a rescuer with the mobile information terminal 201 put on the chest of a patient in a stationary state with a substantially constant period and substantially constant compression depth, with the result that in the mobile information terminal 201, the acceleration acquired from the acceleration sensor 18 exhibits a waveform substantially periodically fluctuating, the velocity obtained by integrating the acceleration exhibits a waveform fluctuating in positive and negative directions centering around 0 [m/s], and the distance obtained by this velocity exhibits a waveform periodically reciprocating in one direction starting from 0 [mm].

Thus, it can be considered that when the velocity has a tendency significantly different from a value representing only a movement associated with the compression operation (for example, when the velocity continuously increases or decreases), the gravitational acceleration component or various noise has not completely been removed from the acceleration value acquired from the acceleration sensor 18, and the influence thereof appears. Thus, when the velocity has a tendency of increasing or decreasing, the mobile information terminal 201 performs correction processing such as the velocity tilt correction processing to thereby satisfactorily remove only a part corresponding to this “tendency”.

In other respects as well, the mobile information terminal 201 according to the second embodiment can exert the same functions and effects as those in the first embodiment.

With the above configuration, the mobile information terminal 201 according to the second embodiment performs the gravity correction processing for the acceleration acquired from the acceleration sensor 18, performs the velocity tilt correction processing after calculating the velocity through integration processing, and calculates the moving distance from the extracted start and end points as the compression depth. Then, the mobile information terminal 201 notifies the rescuer who is performing cardiac massage of the highly accurate compression depth in the immediately preceding compression operation so as to allow him or her to perform the cardiac massage with an adequate compression depth.

### 3. Third Embodiment

A mobile information terminal 301 (FIGS. 1 and 2) according to a third embodiment differs from the mobile information terminal 1 according to the first embodiment in that it has a control part 311 and a storage part 312 in place of the control part 11 and storage part 12. Other configurations are the same as those of the mobile information terminal 1 according to the first embodiment.

As in the first and second embodiments, the control part 311 includes a CPU 21, a ROM 22 and a RAM 23 and is configured to read out various programs from the storage part 312 and execute the readout programs. The storage part 312 stores a moving distance calculation program partly different from those of the first and second embodiments.

### 3-1. Calculation of Moving Distance

In the second embodiment, the mobile information terminal 201 subtracts the gravity correction value from the acceleration value acquired from the acceleration sensor 18 and performs the velocity tilt correction processing for the velocity obtained by integrating the resultant acceleration to remove the gravitational acceleration component and residual part of the drift component.

However, as described above, it is known that the gravitational acceleration value slightly varies from day to day and from location to location. Thus, only performing the gravity correction processing and velocity tilt correction processing may fail to completely remove the drift component. In this case, as illustrated in FIG. 13, the local maximum and minimum values do not appear clearly in the waveform of the distance, so that the mobile information terminal 201 may fail to adequately calculate the moving distance.

To cope with this, the mobile information terminal 301 according to the third embodiment has a moving distance calculation program partly different from that of the second embodiment and is configured to perform center correction processing (details will be described later) with respect to the velocity in addition to the above-described gravity correction processing with respect to the acceleration using the gravity correction value and the velocity tilt correction processing with respect to the velocity.

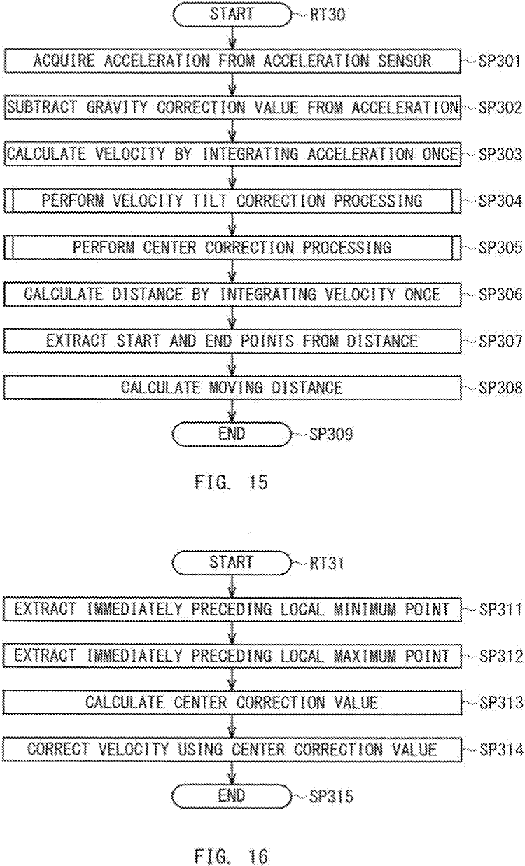

Specifically, when reading out the moving distance calculation program from the storage part 312 and executing the program, the control part 311 of the mobile information terminal 301 forms therein a plurality of functional blocks as illustrated in FIG. 14 corresponding to FIG. 8. The control part 311 forms a center correction part 337 in addition to the acceleration acquisition part 31, gravity correction processing part 32, integration processing part 33, feature point extraction part 34, and moving distance calculation part 35 (which are the same as those of the first embodiment) and velocity tilt correction part 236 (which is the same as that of the second embodiment). The center correction part 337 brings a center value between the local maximum and minimum values in the velocity waveform to 0 [m/s] if it is deviated therefrom (details will be described later).

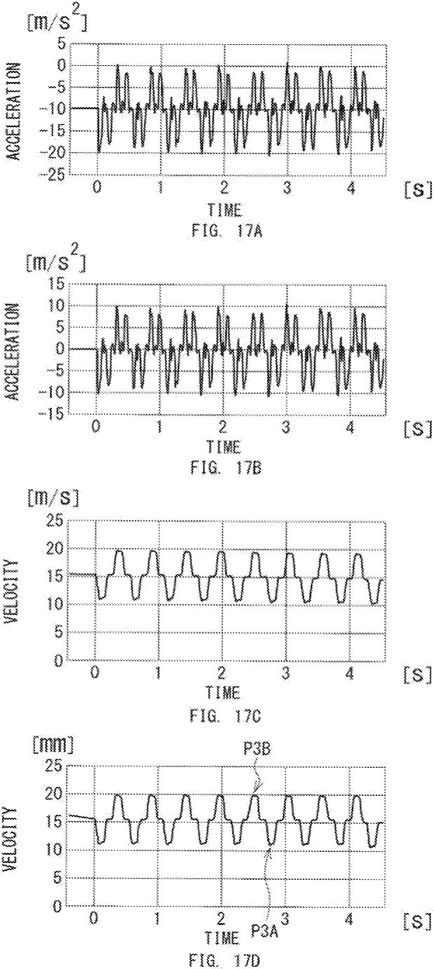

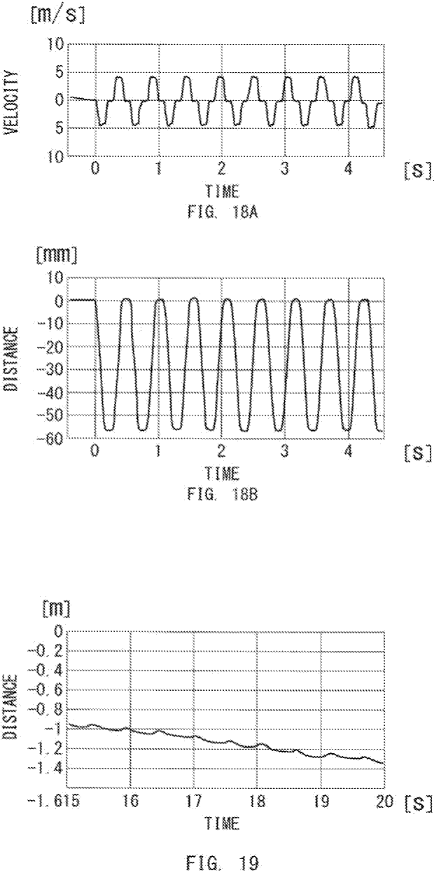

After forming the above functional blocks, the control part 311 starts a moving distance calculation processing routine RT30 illustrated in FIG. 15 corresponding to FIG. 9 and proceeds to step SP301. In steps SP301 to SP304, the control part 311 performs the same processing as those in steps SP201 to SP204 of the moving distance calculation processing routine RT20 (FIG. 9) according to the second embodiment and then proceeds to step SP305. Through this processing, the control part 311 subtracts the gravity correction value from the acceleration illustrated in FIG. 17A to obtain the acceleration illustrated in FIG. 17B, integrates the obtained acceleration to calculate the velocity illustrated in FIG. 17C, and performs the velocity tilt correction processing to obtain the velocity illustrated in FIG. 17D.

In step SP305, the control part 311 performs the center correction processing. A basic approach to the v center correction processing will herein be described. The gravitational acceleration component and drift component have been sufficiently removed from the velocity obtained by the processing up to step SP304; however, it is possible to allow the start and end points in the compression operation to appear more clearly in the distance after integration by bringing the intermediate value between the local maximum and minimum values of the velocity to 0 [m/s].

Thus, in the center correction processing, focusing on the local maximum and minimum values appearing in the velocity waveform in the immediately preceding compression operation, the center value (intermediate value) between the local maximum and minimum values is calculated, followed by subtraction of the calculated center value from the velocity, so as to bring the fluctuation center of the velocity to 0 [m/s].

Specifically, the control part 311 starts a center correction processing routine RT31 illustrated in FIG. 16 as a sub routine and then proceeds to SP311. In step SP311, the control part 311 uses the feature point extraction part 34 (FIG. 14) to extract, from feature points appearing in the velocity waveform (FIG. 17D), an immediately preceding local minimum value, stores the value of the extracted local minimum value, and then proceeds to step SP312.

The feature point extraction part 34 extracts, as the local minimum value, a time point at which the velocity turns from decrease to increase in the same manner as extraction of the local minimum value of the velocity in the first embodiment. That is, the feature point extraction part 34 determines a velocity at a time point when the sign of the acceleration obtained by differentiating the velocity turns from negative to positive as the local minimum value.

In step SP312, the control part 311 uses the feature point extraction part 34 (FIG. 14) to extract, from the feature points appearing in the velocity waveform (FIG. 17D), a local maximum value appearing immediately before the local minimum value extracted in step SP311, stores the extracted local maximum value, and then proceeds to step SP313. The feature point extraction part 34 extracts, as the local maximum value, a time point at which the velocity turns from decrease to increase, specifically, a time point when the sign of the acceleration obtained by differentiating the velocity turns from positive to negative. For example, assuming that the current time is 3 [s] in FIG. 17D, the feature point extraction part 34 extracts points P3A which is the immediately preceding local minimum value and P3B appearing immediately before the local minimum value.

In step SP313, the control part 311 uses the center correction part 337 (FIG. 14) to calculate the intermediate value between the local maximum and minimum values as a center correction value and then proceeds to step SP314.

In step SP314, the control part 311 uses the center correction part 337 (FIG. 14) to subtract the center correction value from the velocity to correct the velocity. Thus, as illustrated in FIG. 18A, the center value of the velocity is brought to 0 [m/s]. Thereafter, the control part 311 proceeds to step SP315 to end the center correction processing routine RT31, then returns to step SP305 in the moving distance calculation processing routine RT30 (FIG. 15), and then proceeds to step SP306.

After that, in steps SP306 to SP308, the control part 311 performs the same processing as those in steps SP205 to SP207 of the moving distance calculation processing routine RT20 (FIG. 9) according to the second embodiment. As a result, the control part 311 calculates the distance exhibiting a waveform illustrated in FIG. 18B, identifies the start and end points in the immediately preceding compression operation, and calculates the moving distance between the start and end points. Thereafter, the control part 311 proceeds to step SP309 to end the moving distance calculation processing routine RT30.

### 3-2. Effects, Etc.

With the above configuration, the mobile information terminal 301 according to the third embodiment executes the moving distance calculation program to subtract the gravity correction value from the acceleration acquired from the acceleration sensor 18 and performs the velocity tilt correction processing for the velocity obtained by integrating the resultant acceleration. Subsequently, the mobile information terminal 301 performs the center correction processing for the resultant velocity to bring the center value between the local maximum and minimum values to 0 [m/s]. Thereafter, as in the second embodiment, the mobile information terminal 301 extracts the start and end points from the distance to calculate the moving distance as the compression depth in the immediately preceding compression operation.

That is, the mobile information terminal 301 performs three stages of the correction processing based on the acceleration acquired from the acceleration sensor 18 incorporated therein to thereby calculate the moving distance with higher accuracy than in the first and second embodiments. Then, as in the first and second embodiments, the mobile information terminal 301 displays the moving distance on the display part 16 as the compression depth to notify the rescuer who is performing cardiac massage of the compression depth in the immediately preceding compression operation in the form of a numeric value so as to allow him or her to perform the cardiac massage with an adequate compression depth.

In particular, by bringing the center value between the local maximum and minimum values to 0 [m/s] after performing the same correction processing as that of the first embodiment and the same velocity tilt correction processing as that of the second embodiment, the mobile information terminal 301 can significantly improve the accuracy of identifying the start and end points of the compression operation based on the distance obtained by the resultant velocity.

In other respects as well, the mobile information terminal 301 according to the third embodiment can exert the same functions and effects as those in the first and second embodiments.

With the above configuration, the mobile information terminal 301 according to the third embodiment subtracts the gravity correction value from the acceleration acquired from the acceleration sensor 18, calculates the acceleration by integration processing, performs the velocity tilt correction processing and center correction processing, calculates the distance by integration processing, and calculates the moving distance from the extracted start and end points as the compression depth. Then, the mobile information terminal 301 notifies the rescuer who is performing cardiac massage of the highly accurate compression depth in the immediately preceding compression operation so as to allow him or her to perform the cardiac massage with an adequate compression depth.

### 4. Fourth Embodiment

A mobile information terminal 401 (FIGS. 1 and 2) according to a fourth embodiment differs from the mobile information terminal 1 according to the first embodiment in that it has a control part 411 and a storage part 412 in place of the control part 11 and storage part 12. Other configurations are the same as those of the mobile information terminal 1 according to the first embodiment.

As in the first and second embodiments, the control part 411 includes a CPU 21, a ROM 22 and a RAM 23 and is configured to read out various programs from the storage part 412 and execute the readout programs. The storage part 412 stores a moving distance calculation program partly different from those of the first to third embodiments.

### 4-1. Calculation of Moving Distance

In the third embodiment, the mobile information terminal 301 subtracts the gravity correction value from the acceleration value acquired from the acceleration sensor 18 and performs the velocity tilt correction processing and center correction processing for the velocity obtained by integrating the resultant acceleration so as to remove the gravitational acceleration component and residual part of the drift component, whereby the local maximum and minimum values can be identified with high accuracy.

However, as described above, it is known that the gravitational acceleration value slightly varies from day to day and from location to location. Thus, only performing the gravity correction processing, velocity tilt correction processing, and center correction processing may fail to completely remove the drift component. In this case, in the distance waveform, a distance value which should return to a certain position every compression operation continues to increase or decrease every time the compression operation is repeated, with the result that the waveform tilts. That is, in this case, the accuracy of the distance to be calculated is reduced, so that the mobile information terminal 301 may fail to adequately calculate the moving distance.

To cope with this, the mobile information terminal 401 according to the fourth embodiment has a moving distance calculation program partly different from that of the third embodiment and is configured to perform distance tilt correction processing (details will be described later) with respect to the distance, in addition to the above-described gravity correction processing with respect to the acceleration using the gravity correction value and the velocity tilt correction processing and center correction processing with respect to the velocity.

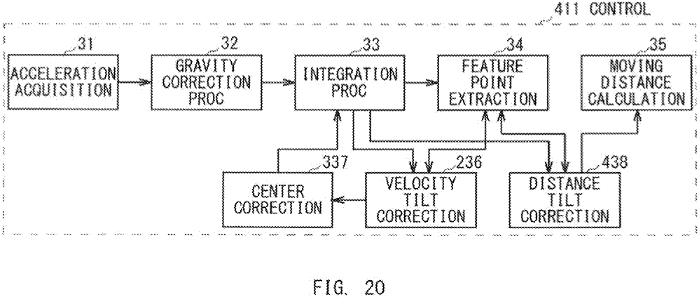

Specifically, when reading out the moving distance calculation program from the storage part 412 and executing the program, the control part 411 of the mobile information terminal 401 forms therein a plurality of functional blocks as illustrated in FIG. 20 corresponding to FIG. 14. The control part 411 forms a distance tilt correction part 438 in addition to the acceleration acquisition part 31, gravity correction processing part 32, integration processing part 33, feature point extraction part 34, and moving distance calculation part 35 (which are the same as those of the first embodiment), velocity tilt correction part 236 (which is the same as that of the second embodiment), and center correction part 337 (which is the same as that of the third embodiment). The distance tilt correction part 438 performs correction to eliminate a distance tilt. Specifically, when the distance waveform has a tendency of tilting due to the drift component, that is, the distance has a tendency of increasing or decreasing with lapse of time, the distance tilt correction part 438 eliminates the distance tilt (details will be described later).

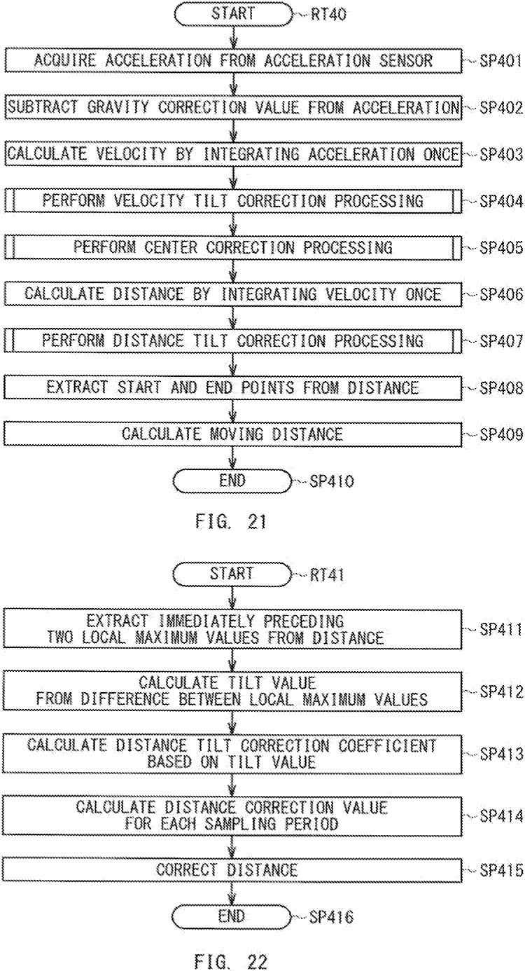

After forming the above functional blocks, the control part 411 starts a moving distance calculation processing routine RT40 illustrated in FIG. 21 corresponding to FIG. 15 and proceeds to step SP401. In steps SP401 to SP406, the control part 411 performs the same processing as those in steps SP301 to SP306 of the moving distance calculation processing routine RT30 (FIG. 15) according to the third embodiment and then proceeds to step SP307.

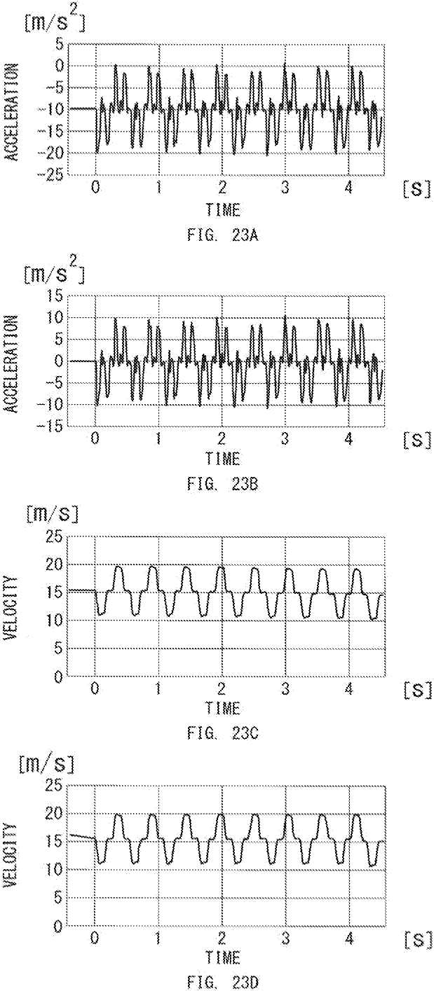

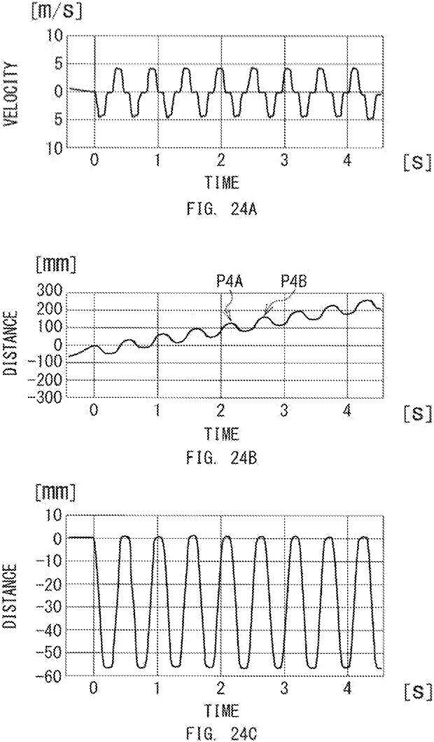

Through this processing, the control part 411 subtracts the gravity correction value from the acceleration illustrated in FIG. 23A to obtain the acceleration illustrated in FIG. 23B and integrates the obtained acceleration to calculate the velocity illustrated in FIG. 23C. Subsequently the control part 411 performs the velocity tilt correction processing for the calculated velocity to obtain the velocity illustrated in FIG. 23D. Further, the control part 411 performs the center correction processing to obtain the velocity illustrated in FIG. 24A and then integrates this velocity to calculate the distance illustrated in FIG. 24B.

In step SP407, the control part 411 performs the distance tilt correction processing. The control part 411 performs the same processing as the velocity tilt correction processing described in the second embodiment for the distance to thereby correct the distance.

Specifically, the control part 411 starts a distance tilt correction processing routine RT41 illustrated in FIG. 22 as a sub routine and then proceeds to SP411. In step SP411, the control part 411 uses the feature point extraction part 34 (FIG. 20) to extract, from feature points appearing in the distance waveform (FIG. 24B), feature points representing immediately preceding two consecutive local maximum values, determines the extracted feature points as a start feature point and an end feature point (hereinafter, referred to also as “distance start feature point and distance feature end point”) in the order of appearance, and then proceeds to step SP412.

As in the extraction of the local maximum value of the velocity in the second embodiment, the feature point extraction part 34 extracts, as the local maximum value, a velocity at a time point at which the distance turns from increase to decrease Specifically, the feature point extraction part 34 determines a velocity at a time point when the sign of the acceleration obtained by differentiating the velocity turns from positive to negative as the local maximum value. For example, assuming that the current time is 3 [s] in FIG. 24B, the feature point extraction part 34 determines points P4A and P4B which are immediately preceding two consecutive local maximum values as the start feature point and end featured point, respectively, in the order of appearance.

In step SP412, the control part 411 uses the distance tilt correction part 438 (FIG. 20) to calculate differences in distance and time between the start feature point and end feature point as a distance difference and a time difference, respectively and then proceeds to step SP413. In step SP413, the control part 411 uses the distance tilt correction part 438 (FIG. 20) to subtract the distance difference by the time difference to thereby calculate a distance tilt correction coefficient representing the ratio of the distance relative to the time and then proceeds to step SP414.

In step SP414, the control part 411 uses the distance tilt correction part 438 (FIG. 20) to calculate a distance correction value (hereinafter, referred to also as “tilt correction value” or “distance tilt correction value”) for the distance every sampling period in between the start feature point and end feature point and then proceeds to step SP415. Specifically, the control part 411 multiplies an elapsed time from the start feature point to each sampling period by the distance tilt correction coefficient to calculate the distance correction value in each sampling period.

In step SP415, the control part 411 uses the distance tilt correction part 438 (FIG. 20) to subtract the distance correction value in each sampling period from the distance in between the start feature point to end feature point to thereby correct the distance. Further, at this time, the distance tilt correction part 438 subtracts also the distance at the start feature point from the distance in between the start feature point to end feature point.

As a result, as illustrated in FIG. 24C, the distances at the start feature point and end feature point have the same value after correction, and increase or decrease of the distance due to the drift component is eliminated. Further, after correction, the distances at the start feature point and at the end feature point both have a value of 0 [m/s].

After that, the control part 411 proceeds to step SP416 to end the distance tilt correction processing routine RT41, then returns to step SP407 in the moving distance calculation processing routine RT40 (FIG. 21), and then proceeds to step SP408.

After that, in steps SP408 and SP409, the control part 411 performs the same processing as those in steps SP307 and SP308 of the moving distance calculation processing routine RT30 (FIG. 15) according to the third embodiment. As a result, the control part 411 identifies the start and end points in the immediately preceding compression operation based on the distance whose waveform is illustrated in FIG. 24C and calculates the moving distance between the start and end points. Thereafter, the control part 411 proceeds to step SP401 to end the moving distance calculation processing routine RT40.

FIGS. 23A to 23D and FIGS. 24A to 24C illustrate the waveform of the acceleration obtained from the acceleration sensor 18 and waveforms of the velocity and distance obtained based on the acceleration when a dedicated tool (not illustrated) is used to perform the compression operation with the compression depth set to 55 [mm] and compression frequency set to 110 [rpm] as described in the first embodiment.

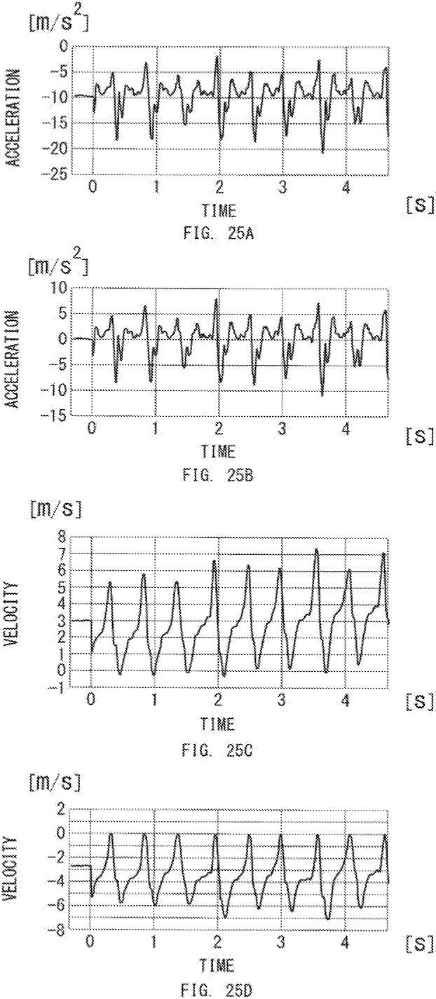

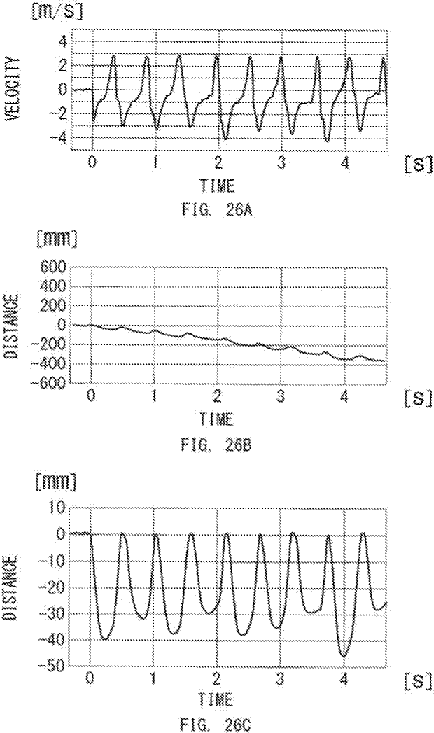

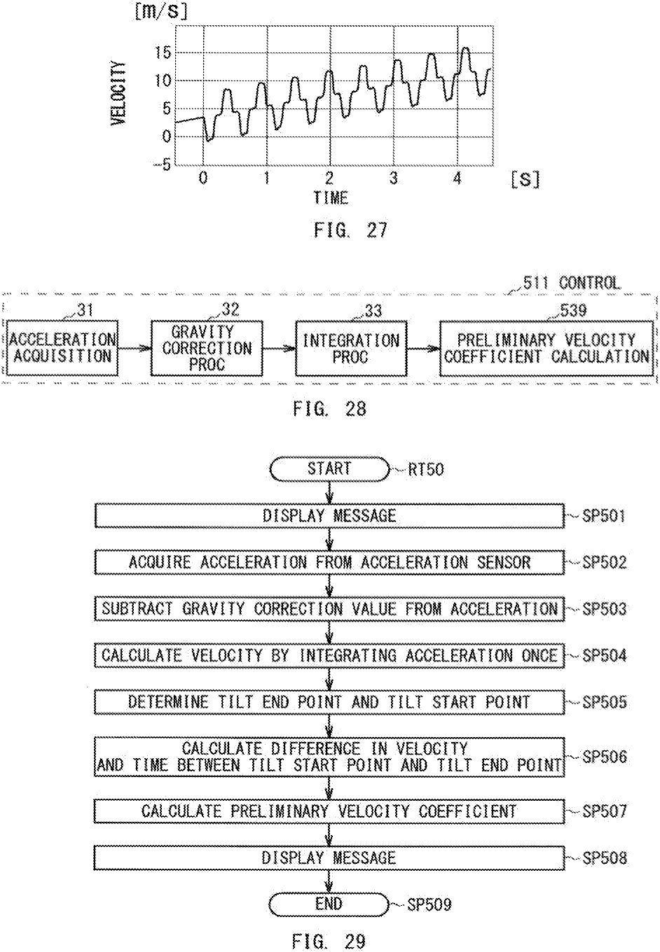

On the other hand, FIGS. 25A to 25D and FIGS. 26A to 26C corresponding to FIGS. 23A to 23D and FIGS. 24A to 24C illustrate the waveform of the acceleration acquired from the acceleration sensor 18 and waveforms of the velocity and distance obtained based on the acceleration when a rescuer performs training of cardiopulmonary resuscitation for a doll for cardiopulmonary resuscitation training without using the dedicated tool. As can be seen from FIGS. 25A to 25D and FIGS. 26A to 26C, the mobile information terminal 401 can finally obtain a satisfactory distance waveform even in the actual cardiopulmonary resuscitation and can thus calculate the moving distance with high accuracy.

### 4-2. Effects, Etc.

With the above configuration, the mobile information terminal 401 according to the fourth embodiment executes the moving distance calculation program to subtract the gravity correction value from the acceleration acquired from the acceleration sensor 18 and performs the velocity tilt correction processing and center correction processing for the velocity obtained by integrating the resultant acceleration. Subsequently, the mobile information terminal 401 performs the distance tilt correction processing for the distance obtained by integrating the resultant velocity to remove the influence of the velocity drift component. Thereafter, as in the third embodiment, the mobile information terminal 401 extracts the start and end points from the distance to calculate the moving distance as the compression depth in the immediately preceding compression operation.

That is, the mobile information terminal 401 performs four stages of the correction processing based on the acceleration acquired from the acceleration sensor 18 incorporated therein to thereby calculate the moving distance with higher accuracy than in the first, second, and third embodiments. Then, as in the first, second, and third embodiments, the mobile information terminal 401 displays the moving distance on the display part 16 as the compression depth to notify the rescuer who is performing cardiac massage of the compression depth in the immediately preceding compression operation in the form of a numeric value so as to allow him or her to perform the cardiac massage with an adequate compression depth.

In particular, with the distance tilt correction processing, the mobile information terminal 401 can sufficiently remove a part of the drift component that has not been removed by the correction processing using the gravity correction value, velocity tilt correction processing, and center correction processing which are the same processing as in the third embodiment and thus can significantly improve the calculation accuracy of the distance as compared to the third embodiment.

Further, in the distance tilt correction processing, as in the velocity tilt correction processing according to the second embodiment, the mobile information terminal 401 determines the two consecutive local maximum values appearing in the distance waveform as the start feature point and end feature point, regards the velocity drift component included in the velocity based on which the distance is acquired as constant in a time period of about 0.5 [s] corresponding to between the start feature point and end feature point, and treats it as one increasing or decreasing at a constant tilt angle in the distance.

Thus, as in the second embodiment, the mobile information terminal 401 can satisfactorily correct a part corresponding to the drift component while significantly reduce a processing load required for computation as compared to a case where the drift component is treated as a constantly varying value. As a result, as in the second embodiment, the mobile information terminal 401 which is a smartphone can calculate, in a range within which real time computation can be achieved by the computation processing capability of the control part 411, a highly accurate distance from which the velocity drift component has effectively been removed.

In the mobile information terminal 401, as illustrated in FIGS. 25A to 25D and FIGS. 26A to 26C, when the acceleration obtained in the case where the rescuer performs the compression operation is used a base, a difference degree in the waveform between one compression operation and another is larger than that in the case (FIGS. 23A to 23D and FIGS. 24A to 24C) where a dedicated tool is used perform the compression operation. The mobile information terminal 401 performs various correction processing mainly based on the acceleration value obtained in the immediately preceding compression operation, so that when the waveform in one compression operation and waveform in another compression operation differ significantly, the correction processing may fail to be performed satisfactorily.

However, as illustrated in FIGS. 25A to 25D and FIGS. 26A to 26C, even when the acceleration obtained in the case where the rescuer performs the compression operation is used a base, a distance value returns to about 0 [mm] for each compression operation in the finally obtained distance waveform (FIG. 26C), and the mobile information terminal 401 can calculate the compression depth (i.e., moving distance) with high accuracy. That is, even when the waveform slightly differ between in one compression operation and another in the compression operation performed by the rescuer, the mobile information terminal 401 can satisfactorily remove an unnecessary component due to the gravitational acceleration by the four stages of correction processing performed based on the acceleration in the immediately preceding compression operation.

In other respects as well, the mobile information terminal 401 according to the fourth embodiment can exert the same functions and effects as those in the first, second, and third embodiments.

With the above configuration, the mobile information terminal 401 according to the fourth embodiment subtracts the gravity correction value from the acceleration acquired from the acceleration sensor 18, calculates the acceleration by integration processing, performs the velocity tilt correction processing and center correction processing, and calculates the distance by integration processing. Subsequently, the mobile information terminal 401 performs the distance tilt correction processing for the calculated distance and calculates the moving distance from the extracted start and end points as the compression depth. Then, the mobile information terminal 401 notifies the rescuer who is performing cardiac massage of the highly accurate compression depth in the immediately preceding compression operation so as to allow him or her to perform the cardiac massage with an adequate compression depth.

### 5. Fifth Embodiment

A mobile information terminal 501 (FIGS. 1 and 2) according to a fifth embodiment differs from the mobile information terminal 1 according to the first embodiment in that it has a control part 511 and a storage part 512 in place of the control part 11 and storage part 12. Other configurations are the same as those of the mobile information terminal 1 according to the first embodiment.

As in the first and second embodiments, the control part 511 includes a CPU 21, a ROM 22 and a RAM 23 and is configured to read out various programs from the storage part 512 and execute the readout programs. The storage part 512 stores a moving distance calculation program partly different from those of the first to fourth embodiments.

As described above, it is known that the gravitational acceleration value slightly varies from day to day and from location to location. That is, as described in the second embodiment, a component due to gravitational acceleration is included in the acceleration acquired from the acceleration sensor 18. Thus, if the correction processing using the gravity correction value is performed for the acceleration acquired from the acceleration sensor 18 in a stationary state before start of the compression operation, and then integration processing is performed for the resultant acceleration to calculate the velocity as in the first embodiment, the calculated velocity does not become 0 [m/s] but may have a waveform exhibiting an increase or a decrease as illustrated in FIG. 27.

Thus, the mobile information terminal 501 is configured to perform two processing stages of preliminary velocity coefficient calculation processing and moving distance calculation processing. In the preliminary velocity coefficient calculation processing of the former stage, a preliminary velocity coefficient is calculated based on the tilt of the velocity acquired in a stationary state before start of the compression operation.

Specifically, when reading out the moving distance calculation program from the storage part 512 and executing the program, the mobile information terminal 501 forms therein a plurality of functional blocks as illustrated in FIG. 28 corresponding to FIG. 4. The control part 511 forms a preliminary velocity coefficient calculation part 539 in addition to the acceleration acquisition part 31, gravity correction processing part 32, and integration processing part 33 (which are the same as those of the first embodiment).

The preliminary velocity coefficient calculation part 539 is partly similar to the velocity tilt correction part 236 (FIG. 8) according to the second embodiment. That is, when the velocity waveform has a tendency of tilting, i.e., the velocity has a tendency of increasing or decreasing with lapse of time, the preliminary velocity coefficient calculation part 539 calculates a preliminary velocity coefficient based on the tilt (details will be described later).

After forming the above functional blocks, the control part 511 starts a preliminary velocity coefficient calculation processing routine RT50 illustrated in FIG. 29 corresponding to FIG. 9 and proceeds to step SP501. In steps SP501, the control part 511 displays a message like “Preliminary processing is performed. Stop moving your smartphone.” on the display part 16 (FIG. 2) to urge the rescuer to bring the mobile information terminal 501 into a stationary state and then proceeds to step SP502.

In steps SP502 to SP504, the control part 511 performs the same processing as those in steps SP201 to SP203 of the moving distance calculation processing routine RT20 (FIG. 9) according to the second embodiment and then proceeds to step SP505.

In step SP505, the control part 511 uses the preliminary velocity coefficient calculation part 539 to set, based on the velocity, one second before the current time as a tilt end point and one second before the tilt end point as a tilt start point and then proceeds to step SP506.

In step SP506, the control part 511 uses the preliminary velocity coefficient calculation part 539 (FIG. 26) to calculate differences in velocity and time between the tilt start point and tilt end point as a velocity difference and a time difference, respectively and then proceeds to step SP507. In step SP507, the control part 511 uses the preliminary velocity coefficient calculation part 539 (FIG. 26) to subtract the velocity difference by the time difference to thereby calculate a preliminary velocity coefficient representing the ratio of the velocity relative to the time, stores the calculated preliminary velocity coefficient in the storage part 512, and then proceeds to step SP508.

In step SP508, the control part 511 displays a message like “Preliminary processing is ended. Start compression operation.” on the display part 16 (FIG. 2) to urger the rescuer to start the compression operation. After that, the control part 511 proceeds to step SP509 to end the preliminary velocity coefficient calculation processing routine RT50.

On the other hand, in the moving distance calculation processing of the latter stage, the processing similar to the moving distance calculation processing routine RT30 (FIG. 15) according to the third embodiment is performed. That is, the correction processing is performed using the preliminary velocity coefficient, followed by the center correction processing and distance tilt correction processing, to thereby calculate the moving distance.

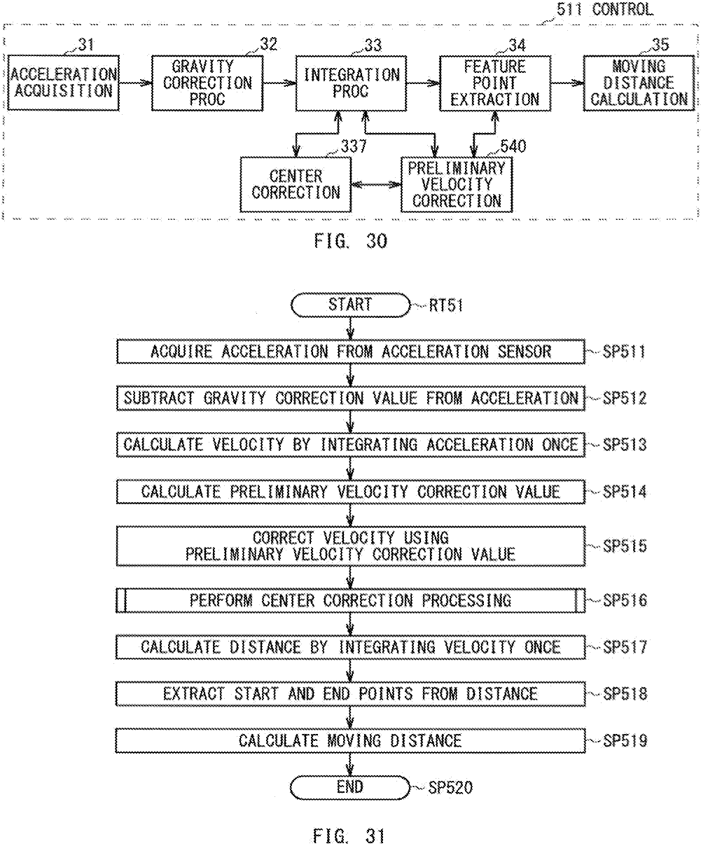

Specifically, when reading out the moving distance calculation program from the storage part 512 and executing the program, the control part 511 forms therein a plurality of functional blocks as illustrated in FIG. 30 corresponding to FIG. 20. The control part 511 forms, in addition to the acceleration acquisition part 31, gravity correction processing part 32, integration processing part 33, feature point extraction part 34, moving distance calculation part 35, and center correction part 337 (which are the same as those of the third embodiment), a preliminary velocity correction part 540 which is formed in place of the velocity tilt correction part 236. The preliminary velocity correction part 540 calculates a preliminary velocity correction value based on the preliminary velocity coefficient and an elapsed time and uses the calculated preliminary velocity correction value to correct the velocity.

After forming the above functional blocks, the control part 511 starts a moving distance calculation processing routine RT51 illustrated in FIG. 31 and proceeds to step SP511. In steps SP511 to SP513, the control part 511 performs the same processing as those in steps SP301 to SP303 (FIG. 15) and then proceeds to step SP514.

In step SP514, the control part 511 uses the preliminary velocity correction part 540 to calculate the preliminary velocity correction value and then proceeds to step SP515. Specifically, the preliminary velocity correction part 540 reads out the preliminary velocity coefficient from the storage part 512 and uses the clocking part 14 (FIG. 2) to acquire a time elapsed from the start of the compression operation. Then, the preliminary velocity correction part 540 multiplies the elapsed time by the preliminary velocity coefficient to thereby calculate the preliminary velocity correction value.

In step SP515, the control part 511 uses the preliminary velocity correction part 540 to subtract the preliminary velocity correction value from the velocity to thereby correct the velocity and then proceeds to step SP516. In steps SP516 to SP519, the control part 511 performs the same processing as those in steps SP305 to SP308 (FIG. 15) and then proceeds to step SP520 to end the moving distance calculation processing routine RT51.

Thus, as in the third embodiment, the mobile information terminal 501 performs the preliminary velocity correction processing to make it possible to sufficiently remove a part of the drift component that has not been removed by the correction processing using the gravity correction value and further performs the center correction processing, whereby it is possible to significantly improve the calculation accuracy of the distance as compared to the first embodiment.

In particular, the mobile information terminal 501 calculates the preliminary velocity coefficient before start of the compression operation, so that it is possible to significantly reduce a processing load required for computation to be performed after start of the compression operation as compared to the third embodiment and thus to calculate the moving distance at an adequate timing with no delay from the compression operation.

### 6. Sixth Embodiment

A mobile information terminal 601 (FIGS. 1 and 2) according to a sixth embodiment differs from the mobile information terminal 1 according to the first embodiment in that it has a control part 611 and a storage part 612 in place of the control part 11 and storage part 12. Other configurations are the same as those of the mobile information terminal 1 according to the first embodiment.

As in the first embodiment, the control part 611 includes a CPU 21, a ROM 22 and a RAM 23 and is configured to read out various programs from the storage part 612 and execute the readout programs. The storage part 612 stores a moving distance calculation program partly different from those of the first to fifth embodiments.

The acceleration sensor 18 (FIG. 2) is suspended by a plurality of elastic bodies. Thus, the acceleration sensor 18 has characteristics (so-called frequency characteristics) according to frequency in reciprocating movement.

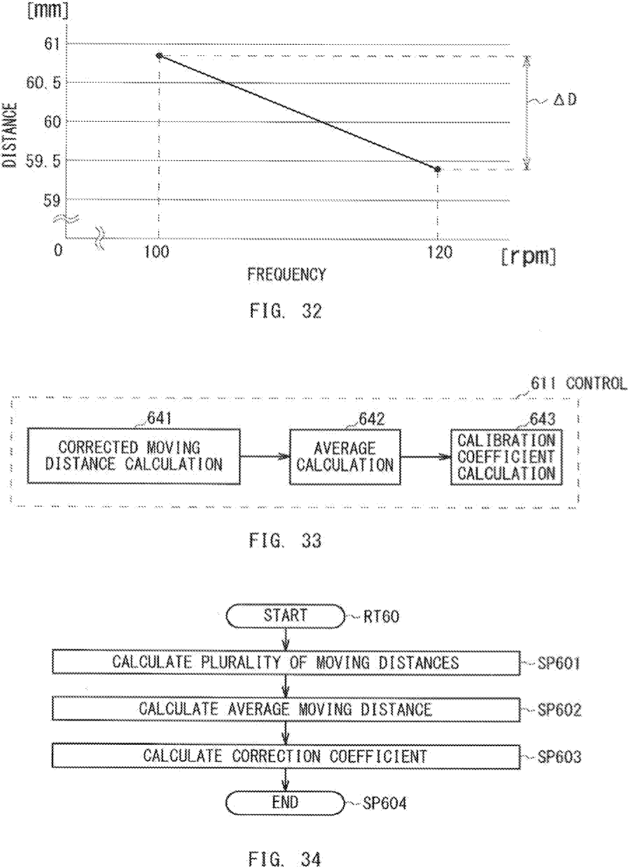

Here, the above-mentioned dedicated tool is used to perform the compression operation with the compression depth set to 60 [mm] and compression frequency set to 100 [rpm] and 120 [rpm]. Under the above conditions, the mobile information terminal 601 is used to calculate the compression depth through various correction processing based on the acceleration acquired from the acceleration sensor 18 as in the fourth embodiment. As a result, as illustrated in FIG. 32, a difference of ΔD (about 1.4 [mm]) occurs between the calculated compression depth value in the case of 100 [rpm] and that in the case of 120 [rpm]. This means that the actual compression depth (60 [mm]) has an error of about 1 [%] or more.

Thus, in this sixth embodiment, the dedicated tool is used to perform the compression operation at a prescribed frequency by a prescribed moving distance, and a ratio between a moving distance calculated at this time and the prescribed moving distance is stored as a calibration coefficient, followed by calibration by multiplication of the distance to be calculated in the subsequent compression operation by the calibration coefficient.

The reason for using the term “calibration” is that when the mobile information terminal 601 is regarded as a “measurement instrument for measuring moving distance”, processing of correcting a calculated value to a correct moving distance specified by the dedicated tool corresponds to so-called “calibration”.

In this sixth embodiment, the mobile information terminal 601 is configured to perform two processing stages of calibration coefficient calculation processing and calibration coefficient application processing. In the calibration coefficient calculation processing of the former stage, the dedicated tool is used to perform the compression operation to calculate the compression depth, and the calibration coefficient is calculated based on the ratio between the obtained calculation result and actual compression depth.

Upon reception of a predetermined operation instruction through the operating part 15 (FIG. 2), the control part 611 of the mobile information terminal 601 forms therein a plurality of functional blocks as illustrated in FIG. 33 when reading out a calibration coefficient calculation program from the storage part 612 and executing it. Specifically, the control part 611 forms a corrected moving distance calculation part 641, an average calculation part 642, and a calibration coefficient calculation part 643.

The corrected moving distance calculation part 641 has therein all the functional blocks (FIG. 20) provided in the fourth embodiment and is configured to perform various correction processing based on the acceleration acquired from the acceleration sensor 18 according to the same method as that of the fourth embodiment to calculate the distance. The average calculation part 642 calculates an average moving distance which is an average value of a plurality of moving distances. The calibration coefficient calculation part 643 calculates the calibration coefficient based on the set compression depth and the average moving distance.

After forming the above functional blocks, the control part 611 starts a calibration coefficient calculation processing routine RT60 illustrated in FIG. 34 and then proceeds to step SP601. In step SP601, in a plurality (e.g., ten times or more) of the compression operations using the dedicated tool, the control part 11 uses the corrected moving distance calculation part 641 to calculate the moving distances based on the respective accelerations acquired from the acceleration sensor 18, stores the calculated moving distances in the storage part 612, and then proceeds to step SP602. For descriptive convenience, the acceleration, velocity, and moving distance obtained here are referred to also as “calibration acceleration”, “calibration velocity”, and “calibration moving distance”. Further, the gravity correction processing performed this time is referred to also as “calibration gravity correction processing”.

At this time, in the dedicated tool, the prescribed compression depth value (55 [mm]) and prescribed frequency value (110 [rpm]) are center values or values close to the center value in proper ranges of the respective compression depth and frequency described in Non-Patent Document 2. Further, at this time, the mobile information terminal 601 performs correction processing for the acceleration value and velocity and distance obtained by integrating the acceleration value according to the moving distance calculation processing routine RT40 (FIG. 21) according to the fourth embodiment so as to perform the subsequent calculation of each moving distance.

In step SP602, the control part 611 uses the average calculation part 642 to calculate the average value of the calculated moving distances as the average moving distance and then proceeds to step SP603. The average moving distance calculated at this time includes a certain error with respect to the prescribed compression depth of 55 [mm] due to the frequency characteristics of the acceleration sensor 18.

In step SP603, the control part 611 uses the calibration coefficient calculation part 643 to subtract the prescribed compression depth (55 [mm]) by the calculated average distance to calculate the calibration coefficient and stores it in the storage part 612. After that, the control part 611 proceeds to step SP604 to end the calibration coefficient calculation processing routine RT60.

On the other hand, in the calibration coefficient application processing of the latter stage, when the mobile information terminal 601 is used to perform the compression operation, the moving distance is calculated based on the acceleration acquired from the acceleration sensor 18, followed by multiplication of the calculation result by the calibration coefficient. Specifically, the control part 611 executes the moving distance calculation processing routine RT40 (FIG. 21) as a sub routine as in the fourth embodiment to calculate the moving distance and stores the moving distance in the storage part 612.

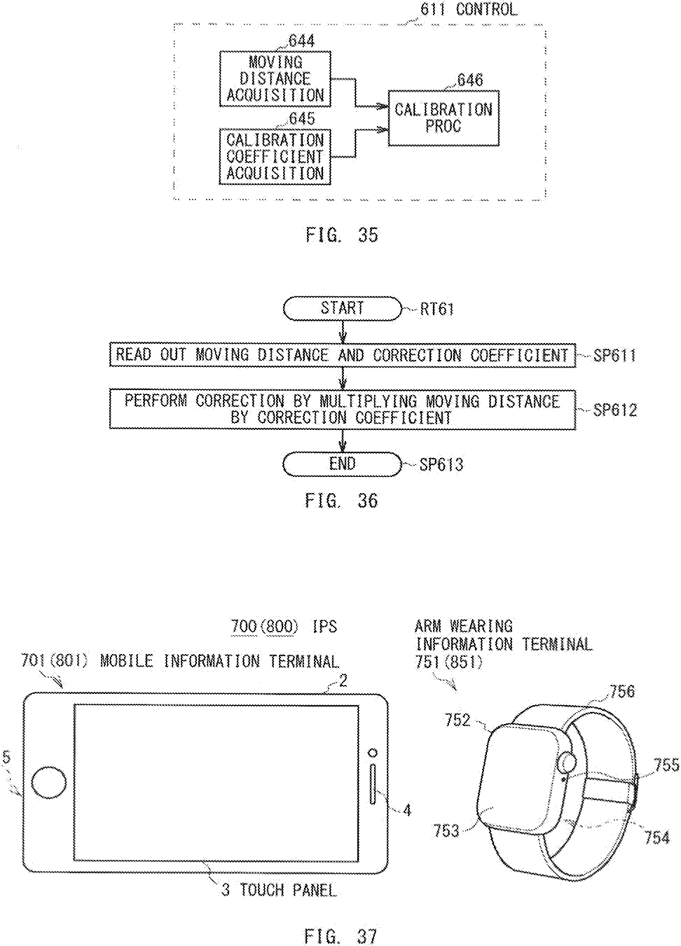

Subsequently, when reading out a calibration coefficient application program from the storage part 612 and executing it, the control part 611 forms therein a plurality of functional blocks as illustrated in FIG. 35. At this time, the control part 611 forms a moving distance acquisition part 644, a calibration coefficient acquisition part 645, and a calibration processing part 646. The moving distance acquisition part 644 reads out the moving distance from the storage part 612 for acquisition. The calibration coefficient acquisition part 645 reads out the calibration coefficient from the storage part 612 for acquisition. The calibration processing part 646 multiplies the moving distance by the calibration coefficient to calibrate the moving distance.

After forming the above functional blocks, the control part 611 starts a calibration coefficient application processing routine RT61 illustrated in FIG. 36 and then proceeds to step SP611. In steps SP611, the control part 511 uses the moving distance acquisition part 644 and calibration coefficient acquisition part 645 to read out the moving distance and correction coefficient and then proceeds to step SP612. In step SP612, the control part 611 uses the calibration processing part 646 to multiply the moving distance by the calibration coefficient to calculate a calibrated moving distance and stores it in the storage part 612. Then, the control part 611 proceeds to step SP613 to end the calibration coefficient application processing routine RT61.

In this manner, the mobile information terminal 601 previously calculates the calibration coefficient based on the acceleration acquired in the compression operation using the dedicated tool and then performs calibration processing by multiplying the moving distance calculated based on the acceleration acquired from the acceleration sensor 18 in the subsequent actual compression operation. As a result, the mobile information terminal 601 can correct an error according to the frequency characteristics of the acceleration sensor 18.

Non-Patent Document 2 describes that an appropriate range of the compression depth is 50 [mm] to 60 [mm] and an appropriate range of the frequency is 100 [rpm] to 120 [rpm]. Correspondingly, in the calibration coefficient calculation processing of the former stage, the mobile information terminal 601 sets the prescribed compression depth to the center value (55 [mm]) of the above range and sets the prescribed frequency to the center value (110 [rpm]) of the above range.

Thus, the mobile information terminal 601 can calculate the moving distance with extremely high accuracy when the compression depth and compression frequency are close to the center values in the above respective ranges in the compression operation performed by the rescuer. Further, the mobile information terminal 601 can suppress a positive and negative error to about 0.5 [%] at a maximum even though the moving distance and frequency are close to the boundary values in the above respective ranges in the compression operation performed by the rescuer.

Further, when calculating the calibration coefficient, the mobile information terminal 601 calculates the average distance using the moving distance obtained by applying the same various correction processing as those of the fourth embodiment. Thus, the mobile information terminal 601 can perform the calibration processing with extremely high accuracy by applying the calibration coefficient to the moving distance calculated by applying the same various correction processing as those of the fourth embodiment in the subsequent actual compression operation.

### 7. Seventh Embodiment

As illustrated in FIG. 37, an information processing system 700 according to a seventh embodiment includes a mobile information terminal 701 and an arm wearing information terminal 751. The mobile information terminal 701 as a second information processing device differs from the mobile information terminal 1 according to the first embodiment in that it has a control part 711, a storage part 712, and a communication part 713 in place of the control part 11, storage part 12, and communication part 13, respectively, as illustrated in FIG. 2. Other configurations are the same as those of the mobile information terminal 1 according to the first embodiment.

As in the first to sixth embodiments, the control part 711 includes a CPU 21, a ROM 22 and a RAM 23 and is configured to read out various programs from the storage part 712 and execute the readout programs. The storage part 712 stores a moving distance calculation program partly different from those of the first to sixth embodiments.

As in the first to sixth embodiments, the communication part 713 can perform wireless communication conforming to a mobile communication standard such as 4G or 5G and a wireless LAN standard such as IEEE 802.11a/b/g/n/ac. In addition, the communication part 713 can perform wireless communication conforming also to a standard such as BLE (Bluetooth® Low Energy).

On the other hand, the arm wearing information terminal 751 as a first information processing device is configured to be a so-called smartwatch having a shape similar to a common watch, as can be seen from the outer appearance thereof illustrated in FIG. 37. Specifically, the arm wearing information terminal 751 has a configuration in which a band 756 is attached to a flat rectangular parallelepiped casing 752. The casing 752 incorporates therein various components and has a touch panel 753 on the front surface thereof. Further, a speaker 754, a microphone 755, and the like are incorporated in the casing 752.

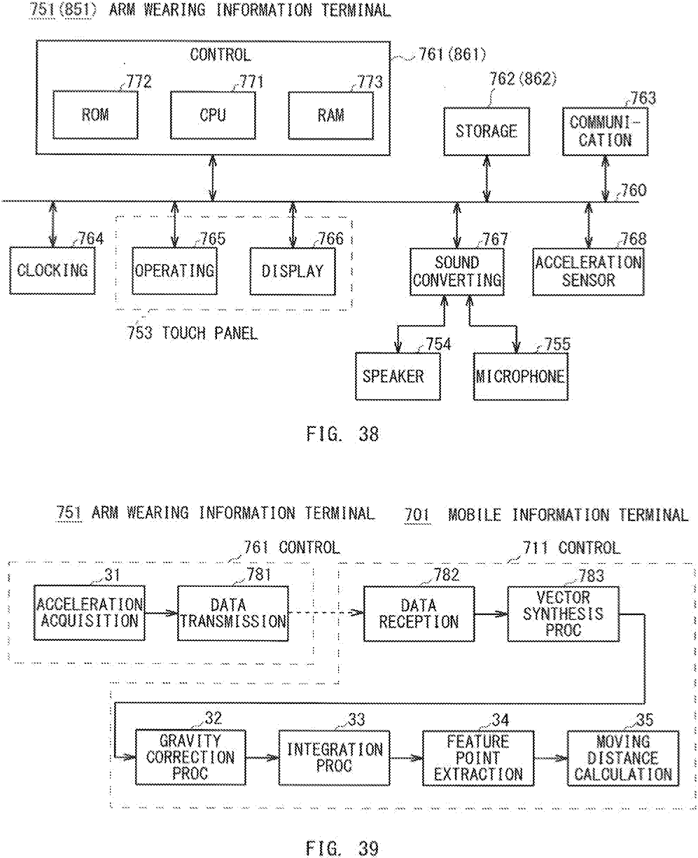

Like the mobile information terminal 701, the arm wearing information terminal 751 is a kind of an information processing device, in which, as illustrated in FIG. 38 corresponding to FIG. 2 and illustrating a schematic circuit configuration, a control part 761, a storage part 762, a communication part 763, a clocking part 764, an operating part 765, a display part 766, a sound converting part 767, and an acceleration sensor 768 are connected through a bus 710. Thus, the arm wearing information terminal 751 has a circuit configuration similar to that of the mobile information terminal 701.

The control part 761 has the same configuration as that of the control part 711 (FIG. 2) of the mobile information terminal 701 and has a CPU 771, a ROM 772, and RAM 773. When a power supply is powered on, the CPU 771 reads out, from the ROM 772 and storage part 762, programs such as an operating system or various applications and executing the readout programs while using the RAM 773 as a work area. The control part 761 thus executes various processing to totally control the arm wearing information terminal 751.

The storage part 762, which is configured similarly to the storage part 712, is constituted by, e.g., a flash memory and stores therein various programs and data. The communication part 763, which is configured similarly to the communication part 713, transmits/receives various information by wireless according to a communication scheme conforming to various communication standards for mobile communication, such as 4G or 5G, and for wireless LAN, such as IEEE 802.11a/b/g/n/ac/ax, and BLE. The clocking part 764, operating part 765, display part 766, sound converting part 767, and acceleration sensor 768 have the same configurations as those of the clocking part 14, operating part 15, display part 16, sound converting part 17, and acceleration sensor 18 (FIG. 2).

The arm wearing information terminal 751 is smaller in volume than the mobile information terminal 701 and thus has a sufficiently small battery capacity. Therefore, the control part 761 of the arm wearing information terminal 751 is reduced in computing power as compared to the control part 711 of the mobile information terminal 701, whereby power consumption can be reduced.

### 7-1. Calculation of Moving Distance

In the first embodiment and other embodiments, the mobile information terminal 1 calculates the moving distance based on the acceleration value acquired from the acceleration sensor 18 provided therein. On the other hand, in the seventh embodiment, the mobile information terminal 701 calculates the moving distance based on the acceleration value acquired from the acceleration sensor 768 of the arm wearing information terminal 751.

Specifically, when reading out an acceleration detection program from the storage part 762 and executing the program, the control part 761 of the arm wearing information terminal 751 forms therein a plurality of functional blocks as illustrated in FIG. 39 corresponding to FIG. 4. Further, when reading out the moving distance calculation program from the storage part 712 and executing the program, the control part 711 of the mobile information terminal 701 forms therein a plurality of functional blocks as illustrated in FIG. 39.

That is, the control part 761 of the arm wearing information terminal 751 forms the same acceleration acquisition part 31 as that formed by the control part 11 according to the first embodiment and a data transmission part 781. Further, the control part 711 of the mobile information terminal 701 forms the gravity correction processing part 32, integration processing part 33, feature point extraction part 34, and moving distance calculation part 35 (which are the same as those of the first embodiment and, in addition thereto, a data reception part 782 and a vector synthesis processing part 783. Specific operations of the respective functional blocks will be described later.

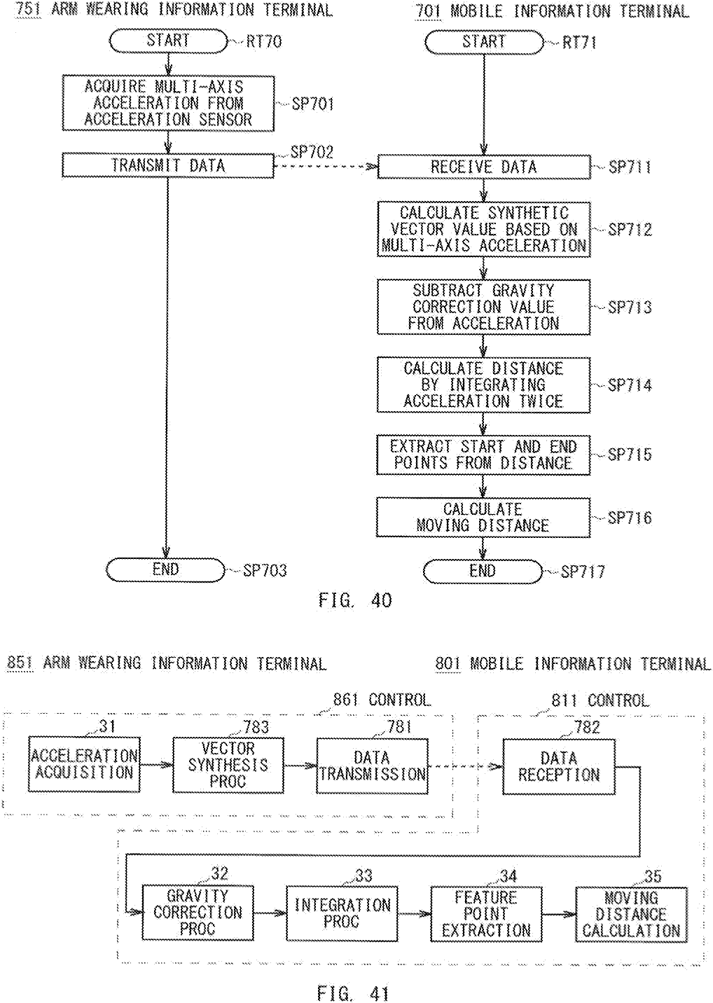

After forming the functional blocks illustrated in FIG. 39, the control part 761 of the arm wearing information terminal 751 starts an acceleration detection processing routine RT70 illustrated in FIG. 40 and then proceeds to step SP701.

In step SP701, the control part 761 performs the same processing as step SP1 of the moving distance calculation processing routine RT1 (FIG. 5) and then proceeds to step SP702. At this time, the acceleration acquisition part 31 (FIG. 39) acquires three-dimensional analog acceleration values from the acceleration sensor 768 of the arm wearing information terminal 751, followed by conversion of the analog values into digital values, thereby acquiring a digital acceleration vector represented by a three-dimensional vector (αX, αY, αZ).

In step SP702, the control part 761 uses the data transmission part 781 (FIG. 39) to transmit, as transmission data, the acceleration vector acquired by the acceleration acquisition part 31 to the mobile information terminal 701 by the BLE. Specifically, the data transmission part 781 generates the transmission data by making the communication part 763 (FIG. 38) apply predetermined conversion processing to the acceleration vector and transmits the generated transmission data by the BLE. Then, the control part 761 proceeds to step SP703 to end the acceleration detection processing routine RT70.

On the other hand, the control part 711 of the mobile information terminal 701 starts a moving distance calculation processing routine RT71 illustrated in FIG. 40 after forming the functional blocks illustrated in FIG. 39 and then proceeds to step SP711.

In step SP711, the control part 711 uses the data reception part 782 (FIG. 39) to take out the original acceleration vector from the transmission data received by the BLE and then proceeds to step SP712. Specifically, the data reception part 782 performs predetermined demodulation processing for the reception data in the communication part 713 (FIG. 2) to restore the original acceleration data and supplies the restored data to the vector synthesis processing part 783.

In step SP712, the control part 711 uses the vector synthesis processing part 783 to calculate, based on the acceleration vector which is the three-dimensional vector value, an acceleration value which is a scholar value corresponding to the magnitude of the acceleration vector and then proceeds to step SP713 . Specifically, the vector synthesis processing part. 783 calculates an acceleration value α by performing computation according to the following expression (1):

$$a = \sqrt{a {X}^{2} + a {Y}^{2} + a {Z}^{2}}$$

After that, in step SP713 to SP716, the control part 711 performs the same processing as steps SP2 to SP5 of the moving distance calculation processing routine RT1 (FIG. 5) and then proceeds to step SP717 to end the moving distance calculation processing routine RT71.

### 7-2. Effects, Etc.

With the above configuration, the information processing system 700 according to the seventh embodiment uses the arm wearing information terminal 751 worn on a user’s wrist to detect the three-dimensional acceleration vector and transmits the obtained data to the mobile information terminal 701. Upon reception of the data from the information processing system 700, the mobile information terminal 701 converts the three-dimensional information the three-dimensional acceleration vector into an acceleration value which is a scholar value, performs the gravity correction processing, performs integration processing twice, and extracts the start and end points to thereby calculate the moving distance as the compression depth in the immediately preceding compression operation.

That is, in the information processing system 700, based on the acceleration acquired from the acceleration sensor 768 of the arm wearing information terminal 751 worn on a user’s wrist, the mobile information terminal 701 can calculate an accurate moving distance. Then, the mobile information terminal 701 displays the moving distance on the display part 16 as the compression depth to notify the rescuer who is performing cardiac massage of the compression depth in the immediately preceding compression operation in the form of a numeric value so as to allow him or her to perform the cardiac massage with an adequate compression depth.

In other respects, in the information processing system 700, unlike the first to sixth embodiments, the acceleration can be detected by the arm wearing information terminal 751, so that it is possible to eliminate the need to put the mobile information terminal 701 between the chest of a patient and the hand of the rescuer and thus to place the mobile information terminal 701 at a position where the rescuer can easily view. Thus, the information processing system 700 allows the rescuer to easily view the display part 16 (i.e., touch panel 3) of the mobile information terminal 701 on which the latest compression depth and period are displayed.

Further, in the information processing system 700, the arm wearing information terminal 751 is lower in processing power and smaller in battery capacity than the mobile information terminal 701. Thus, in the information processing system 700, the arm wearing information terminal 751 takes on only extremely reduced processing of detecting and transmitting the three-dimensional acceleration vector, and the mobile information terminal 701 takes on other processing including computation processing. With this configuration, it is possible to reduce a reduction in operable time without imposing an excessive processing load on the arm wearing information terminal 751.

Further, in the information processing system 700, the moving distance is displayed as the compression depth on the display part 16 (FIG. 2) of the mobile information terminal 701 together with the period. Thus, in the information processing system 700, it is possible to reduce the power consumption of the arm wearing information terminal 751 as compared to when the moving distance and the like is displayed on the display part 766 (FIG. 38) of the arm wearing information terminal 751 and further to allow the rescuer to easily view the display part 16 (i.e., touch panel 3) of the mobile information terminal 701 in a stationary state.

In other respects as well, the information processing system 700 according to the seventh embodiment can exert the same functions and effects as those in the first embodiment.

Thus, the information processing system 700 according to the seventh embodiment is configured such that the arm wearing information terminal 751 acquires the acceleration from the acceleration sensor 768 and that the mobile information terminal 701 performs the gravity correction processing for the acquired acceleration, performs integration processing twice for the resultant acceleration to calculate the distance, and calculates the moving distance from the extracted start and end points as the compression depth. Thus, the information processing system 700 allows the rescuer who is performing cardiac massage to recognize a highly accurate compression depth in the immediately preceding compression operation to thereby allow him or her to perform the cardiac massage with an adequate compression depth.

### 8. Eighth Embodiment

An information processing system 800 (FIG. 37) according to an eighth embodiment includes a mobile information terminal 801 an arm wearing information terminal 851 corresponding respectively to the mobile information terminal 701 and arm wearing information terminal 751 of the information processing system 700 according to the seventh embodiment. As illustrated in FIG. 2, the mobile information terminal 801 differs from the mobile information terminal 701 according to the seventh embodiment in that it has a control part 811, a storage part 812, and a communication part 813 in place of the control part 711, storage part 712, and communication part 713, respectively. Other configurations are the same as those of the mobile information terminal 701 according to the seventh embodiment.

As in the seventh embodiment, the control part 811 includes a CPU 21, a ROM 22 and a RAM 23 and is configured to read out various programs from the storage part 812 and execute the readout programs. The storage part 812 stores a moving distance calculation program partly different from that of the seventh embodiment. The communication part 813, which is configured similarly to the communication part 713 of the seventh embodiment, transmits/receives various information by wireless according to a communication scheme conforming to various communication standards for mobile communication, such as 4G or 5G, and for wireless LAN, such as IEEE 802.11a/b/g/n/ac/ax, and BLE.

The arm wearing information terminal 851 (FIG. 37) differs from the arm wearing information terminal 751 according to the seventh embodiment in that it has a control part 861 and a storage part 862 in place of the control part 761 and storage part 762, respectively. Other configurations are the same as those of the arm wearing information terminal 751 according to the seventh embodiment.

Like the control part 761, the control part 861 includes a CPU 771, a ROM 772 and a RAM 773 and is configured to read out various programs from the storage part 862 and execute the readout programs. The control part 861 executes an acceleration detection program partly different from that of the seventh embodiment.

### 8-1. Calculation of Moving Distance

In this eighth embodiment, as in the seven the embodiment, the mobile information terminal 801 calculates the moving distance based on the acceleration acquired from the acceleration sensor 768 of the arm wearing information terminal 851.

Specifically, when reading out the acceleration detection program from the storage part 862 and executing the program, the control part 861 of the arm wearing information terminal 851 forms therein a plurality of functional blocks as illustrated in FIG. 41 corresponding to FIG. 39. Further, when reading out the moving distance calculation program from the storage part 812 and executing the program, the control part 811 of the mobile information terminal 801 forms therein a plurality of functional blocks as illustrated in FIG. 41.

That is, the control part 861 of the arm wearing information terminal 851 differs from the arm wearing information terminal 751 according to the seventh embodiment in that it additionally has a vector synthesis processing part 783. On the other hand, the control part 811 of the mobile information terminal 801 differs from the control part 711 according to the seventh embodiment in that it does not have the vector synthesis part. That is, in the eighth embodiment, the vector synthesis part is not provided in the mobile information terminal side but provided in the arm wearing information terminal side.

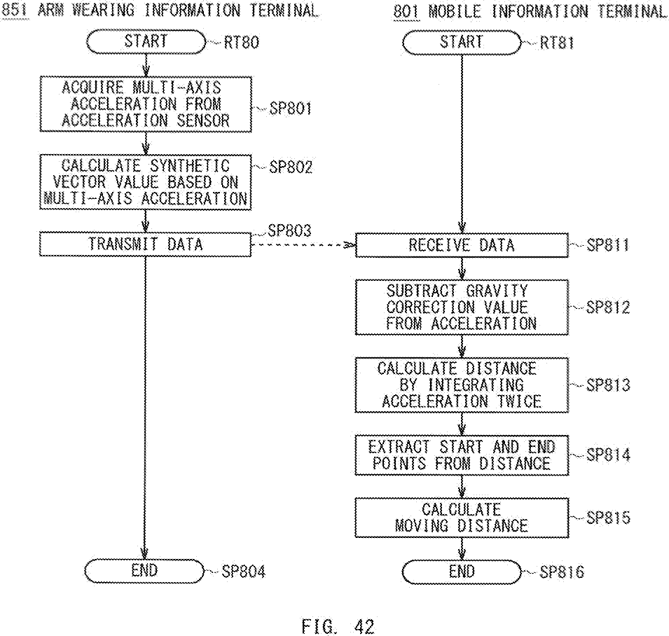

After forming the functional blocks illustrated in FIG. 41, the control part 861 of the arm wearing information terminal 851 starts an acceleration detection processing routine RT80 illustrated in FIG. 42 corresponding to FIG. 40 and then proceeds to step SP801. In step SP801, the control part 861 performs the same processing as step SP701 of the acceleration detection processing routine RT70 (FIG. 39) according to the seventh embodiment to acquire the three-dimensional acceleration vector and then proceeds to step SP802.

In step SP802, as in the step SP712 of the moving distance calculation processing routine RT71, the control part 861 uses the vector synthesis processing part 783 to calculate, based on the acceleration vector which is the three-dimensional vector value, an acceleration value which is a scholar value according to the above expression (1) and then proceeds to step SP803.

In step SP803, as in the step SP702 of the acceleration detection processing routine RT70 (FIG. 39), the control part 861 transmits the transmission data to the mobile information terminal 701 by the BLE. At this time, the control part 861 transmits the acceleration value which is a scholar value calculated by the vector synthesis processing part 783. Then, the control part 861 proceeds to step SP804 to end the acceleration detection processing routine RT70.

On the other hand, the control part 811 of the mobile information terminal 801 starts, after forming the functional blocks illustrated in FIG. 41, a moving distance calculation processing routine RT81 illustrated in FIG. 42 and then proceeds to step SP811. In step SP811, the control part 811 uses the data reception part 782 (FIG. 41) to take out the original acceleration value from the transmission data received by the BLE and then proceeds to step SP812. In steps SP812 to SP815, the control part 811 performs the same processing as steps SP713 to SP716 of the moving distance calculation processing routine RT71 (FIG. 39) and then proceeds to SP816 to end the moving distance calculation processing routine RT81.

### 8-2. Effects, Etc.

With the above configuration, as in the seventh embodiment, the information processing system 800 according to the eighth embodiment uses the arm wearing information terminal 851 worn on a user’s wrist to detect the three-dimensional acceleration vector, convert the detected three-dimensional acceleration vector into an acceleration value which is a scholar value, and transmit the resultant acceleration value to the mobile information terminal 801. The mobile information terminal 801 performs the gravity correction processing for the received acceleration value, performs integration processing twice for the resultant acceleration, and extracts the start and end points to thereby calculate the moving distance as the compression depth in the immediately preceding compression operation.

That is, as in the seventh embodiment, in the information processing system 800, based on the acceleration acquired from the acceleration sensor 768 of the arm wearing information terminal 851 worn on a user’s wrist, the mobile information terminal 801 can calculate an accurate moving distance. Then, the mobile information terminal 801 displays the moving distance on the display part 16 as the compression depth to notify the rescuer who is performing cardiac massage of the compression depth in the immediately preceding compression operation in the form of a numeric value so as to allow him or her to perform the cardiac massage with an adequate compression depth.

In particular, in the information processing system 800, the three-dimensional acceleration vector is previously converted into an acceleration value in the arm wearing information terminal 851, followed by transmission thereof to the mobile information terminal 801, Thus, in the information processing system 800, it is possible to reduce the volume of data to be transmitted from the arm wearing information terminal 851 to mobile information terminal 801 and thus to shorten the time required for calculating the moving distance.

In other respects as well, the information processing system 800 according to the eighth embodiment can exert the same functions and effects as those in the seventh embodiment.

Thus, the information processing system 800 according to the eighth embodiment is configured such that the arm wearing information terminal 851 acquires the acceleration from the acceleration sensor 768 and that the mobile information terminal 801 performs the gravity correction processing for the acquired acceleration, performs integration processing twice for the resultant acceleration to calculate the distance, and calculates the moving distance from the extracted start and end points as the compression depth. Thus, the information processing system 800 allows the rescuer who is performing cardiac massage to recognize a highly accurate compression depth in the immediately preceding compression operation to thereby allow him or her to perform the cardiac massage with an adequate compression depth.

### 9. Other Embodiments

In the above first embodiment, the gravity acceleration value to be used in the correction of the acceleration is set to the value (9.80665 [m/s²]) of the standard gravitational acceleration. However, the present invention is not limited to this, and other various values may be used as the gravitation correction value. For example, the value of an acceleration (hereinafter, referred to also as “gravitational acceleration in stationary state”) detected by the acceleration sensor 18 in a stationary state may be regarded as the gravitational acceleration at this time and location and used as the gravity correction value. The same applies to the second to eighth embodiments.

Further, in the above second embodiment, the velocity tilt correction processing is performed such that two consecutive local maximum values appearing in the velocity waveform are determined as the start and end feature points, followed by calculation of the velocity tilt correction coefficient based on the velocity difference and time difference between the feature points. However, the present invention is not limited to this, and two feature points repeatedly appearing in the velocity waveform may be selected as the start and end feature points. For example, two local maximum values separated so as to interpose therebetween one or more local maximum values in the velocity waveform, or two consecutive local minimum values appearing in the velocity waveform may be determined as the start and end feature points. The same applies to the third to sixth embodiments.

Further, in the above second embodiment, the velocity tilt correction processing is performed such that two local maximum values (or other feature points) appearing in the velocity waveform are determined as the start and end feature points, followed by calculation of the velocity tilt correction coefficient based on the velocity difference and time difference between the feature points. However, the present invention is not limited to this. For example, two local maximum values (or other feature points) appearing in the acceleration waveform may be determined as the start and end feature points, followed by calculation of the acceleration tilt correction coefficient based on the acceleration difference and time difference between the feature points. The same applies to the third to sixth embodiments.

Further, in the above third embodiment, the center correction processing is performed such that the intermediate value between the immediately preceding local maximum value and minimum values is determined as the center value, which is then brought to 0 [m/s] . However, the present invention is not limited to this. For example, an intermediate value between an average local maximum value among a plurality of local maximum values in a plurality of preceding periods and an average local minimum value among a plurality of local minimum values in a plurality of preceding periods may be determined as the center value. Alternatively, focusing on the immediately preceding one period in the velocity waveform, a value at which the areas (i.e., absolute integral values) of positive and negative value zones are equal to each other may be determined as the center value. That is, the center value may be calculated or determined by various methods, followed by bringing of the center value to 0 [m/s]. The same applies to the fourth to sixth embodiments.

Further, in the above third embodiment, the velocity tilt correction processing and center correction processing are performed independently of each other (FIGS. 15 to 18). However, the present invention is not limited to this. For example, in step SP211 of the velocity tilt correction processing (FIG. 10), a point at which the tilt of the velocity, i.e., a value (dV/dt) obtained by differentiating a velocity V with respect to a time t becomes maximum for each time range corresponding to one compression operation may be determined as the feature point. In this case, by bringing the determined featured points to 0 [m/s] when the velocity is corrected based on the velocity tilt correction coefficient in the subsequent step SP215, it is possible to collectively perform the velocity tilt correction processing and center correction processing. The same applies to the fourth to sixth embodiments.

Further, in the above fourth embodiment, the distance tilt correction processing is performed such that two consecutive local maximum values appearing in the distance waveform are determined as the start and end feature points, followed by calculation of the distance tilt correction coefficient based on the distance difference and time difference between the feature points. However, the present invention is not limited to this, and two feature points repeatedly appearing in the distance waveform may be selected as the start and end feature points. For example, two local maximum values separated so as to interpose therebetween one or more local maximum values in the distance waveform, or two consecutive local minimum values appearing in the distance waveform may be determined as the start and end feature points. The same applies to the sixth embodiment.

Further, in the above fourth embodiment, the distance tilt correction processing is performed after completion of the gravity correction processing, velocity tilt correction processing, and center correction processing. However, the present invention is not limited to this. For example, the distance tilt correction processing may be performed after completion of only the gravity correction processing or after completion of only the gravity correction processing and velocity tilt correction processing. The same applies to the fourth to sixth embodiments.

Further, in the above fifth embodiment, the tilt end and start points are set based on the acceleration calculated based on the acceleration, followed by calculation of the stationary velocity tilt correction coefficient, and the velocity is corrected using the stationary velocity tilt correction coefficient in the compression operation. However, the present invention is not limited to this. For example, based on the acceleration (hereinafter, referred to also as “preliminary acceleration”) acquired from the acceleration sensor 18 in a stationary state, an average of the acceleration value between 1 [s] before the current time and 2 [s] before the current time may be calculated as an acceleration correction value for correction of the acceleration in the compression operation. Alternatively, with the velocity waveform between the tilt start and end points regarded as the linear straight line of the velocity with respect to time, an approximate tilt calculated by computation such as least squares may be used as the preliminary velocity coefficient.

Further, in the above fifth embodiment, in a state where the mobile information terminal 501 is kept stationary, 1 [s] before the current time is set as the tilt end point, and 1 [s] before the tilt end point is set as the tilt start point. Then, the preliminary velocity coefficient is calculated based on the velocity difference and time difference between the tilt start and end points. However, the present invention is not limited to this, and other various time points may be set as the tilt end and start points for calculation of the preliminary velocity coefficient. For example, in a state where the mobile information terminal 501 is kept stationary, 0.5 [s] before the current time may be set as the tilt end point, and 2 [s] before the tilt end point may be set as the tilt start point.

Further, in the above sixth embodiment, when the dedicated tool is used to perform the compression operation, the moving distance is calculated with the prescribed compression depth set to 55 [mm] and prescribed compression frequency set to 110 [rpm], and the calibration coefficient is calculated using this moving distance. However, the present invention is not limited to this, and the prescribed compression depth and prescribed compression frequency may be set to various values. For example, the prescribed compression depth may be set to 105 [mm] and prescribed compression frequency may be set to 115 [rpm]. That is, the prescribed compression depth and prescribed compression frequency may be any values as long as they fall within the proper ranges described in Non-Patent Document 2 and are preferably close to the center values of the respective ranges.

Further, in the above sixth embodiment, in the calibration coefficient calculation processing of the former stage, the corrected moving distance calculation part 641 (FIG. 31) performs four correction processing steps (gravity correction processing, velocity tilt correction processing, center correction processing, and distance tilt correction processing) as in the fourth embodiment. However, the present invention is not limited to this, and some of the correction processing steps other than the gravity correction processing may be omitted. In this case, when the moving distance is calculated in the calibration coefficient calculation processing of the latter stage, some of the correction processing steps other than the gravity correction processing are desirably omitted so as to make the correction processing to be applied equivalent.

Further, in the above seventh embodiment (FIG. 39), the acceleration acquisition part 31 is provided on the arm wearing information terminal 751 side, and the vector synthesis processing part 783 and functional blocks including the gravity correction processing part 32 and subsequent parts (33 to 35) are provided on the mobile information terminal 701 side. Further, in the above eighth embodiment (FIG. 41), the acceleration acquisition part 31 and vector synthesis processing part 783 are provided on the arm wearing information terminal 751 side, and the functional blocks including the gravity correction processing part 32 and subsequent parts (33 to 35) are provided on the mobile information terminal 701 side. However, the present invention is not limited to this, and allocation of the functional blocks in the arm wearing information terminal and mobile information terminal may be appropriately changed. For example, the functional blocks including the acceleration acquisition part 31 and gravity correction processing part 32 may be provided on the arm wearing information terminal side, and those including the integration processing part 33 and subsequent parts (34, 35) may be provided on the mobile information terminal side.

Further, in the above seventh embodiment, the arm wearing information terminal 751 is used to detect the acceleration and transmit the detected acceleration to the mobile information terminal 701. However, the present invention is not limited to this, and periodic notification may be performed in accordance with the prescribed compression frequency of 110 [rpm] on the arm wearing information terminal 751 (FIG. 38). For example, a periodically changing display may be made on the display part, or a periodic sound may be output from the speaker 754. Such notification may be performed on the mobile information terminal 701. Further, a vibrator, if provided, in the arm wearing information terminal 751 may be activated in accordance with the prescribed compression frequency of 110 [rpm] . However, in this case, it is desirable to perform correction processing of eliminating the influence of the vibrator on the acceleration value detected by the acceleration sensor 768. The same applies to the eighth embodiment.

Further, in the above seventh embodiment, the transmission data is transmitted/received between the arm wearing information terminal 751 and mobile information terminal 701 using the BLE. However, the present invention is not limited to this, and other various communication schemes, such as wireless LAN conforming to standards such as IEEE 802.11a/b/g/n/ac/ax, and mobile communication standards such as 4G or 5G may be used to perform data transmission/reception.

Further, in the above first embodiment, the moving distance is calculated according to the moving distance calculation program, and then, according to the cardiopulmonary resuscitation program, the moving distance is displayed as the compression depth on the display part (FIG. 2) together with the period so as to notify the rescuer of these information. However, the present invention is not limited to this. For example, the above notification may be performed by display of states of a plurality of stages (e.g., “too shallow”, “appropriate”, and “too deep” for the compression depth, and “too low”, “appropriate”, and “too high” for the compression frequency) . In this case, a graphical display such as a level meter may also be possible. Further, the above notification may be achieved not only by display but also by sound (human voice or sound effect), vibration generated by a vibrator (not illustrated) incorporated in the mobile information terminal 1. Further, these notification means may be appropriately combined. Further, the above notification need not necessarily be performed every compression operation, but may be performed at various timings (e.g., every predetermined number of the compression operations, or every predetermined time).

Further, in the above first embodiment, the acceleration acquisition part 31 and the like (FIG. 4) of the control part 11 are formed as the functional blocks of software. However, the present invention is not limited to this, and at least some of the acceleration acquisition part 31 and the like may be configured as a hardware circuit. The same applies to the second to eighth embodiments.

Further, in the above first embodiment, various application programs including the cardiopulmonary resuscitation program, distance calculation program, and the like are previously stored in the storage part 12 (FIG. 2), and then the applications read out and executed so as to calculate the moving distance. However, the present invention is not limited to this. For example, the various applications may be acquired from an external server or the like (not illustrated) through the communication part 13 for execution. Alternatively, the applications may be read out from a recording medium such as a detachable memory card. That is, the calculation processing of the moving distance and the like may be executed by executing the application programs acquired through various means. The same applies to the second to eighth embodiments.

Further, in the above first embodiment, the present invention is applied to the mobile information terminal 1 which is a smartphone. However, the present invention is not limited to this and may be applied to various information processing devices incorporating an acceleration sensor, such as a tablet terminal, a portable game machine, and a watch-type terminal device called a smartwatch. In this case as well, as in the case where the mobile information terminal 1 is used, the information processing device may be put between the chest of a patient and the hand of a rescuer upon the compression operation. The same applies to the second to eighth embodiments.

The present invention is not limited to the above embodiments and other embodiments but includes an embodiment obtained by arbitrarily combining a part or all of the above embodiments and other embodiments or an embodiment obtained by extracting a part therefrom.

### INDUSTRIAL APPLICABILITY

The present invention may be used when a rescuer performs cardiopulmonary resuscitation for a patient.

REFERENCE SIGNS LIST

| | |
|---|---|
| 1, 201, 301, 401, 501, 601, 701, 801: | Mobile information terminal |
| 11, 211, 311, 411, 511, 611, 711, 811, 761, 861: | Control part |
| 12, 212, 312, 412, 512, 612, 712, 812: | Storage part |
| 13, 713, 813, 763: | Communication part |
| 14: | Clocking part |
| 16, 776: | Display part |
| 18, 768: | Acceleration sensor |
| 31: | Acceleration acquisition part |
| 32: | Gravity correction processing part |
| 33: | Integration processing part |
| 34: | Feature value extraction part |
| 35: | Moving distance calculation part |
| 236: | Velocity tilt correction part |
| 337: | Center correction part |
| 438: | Distance tilt correction part |
| 539: | Preliminary velocity coefficient calculation part |
| 540: | Preliminary velocity correction part |
| 641: | Corrected moving distance calculation part |
| 642: | Average calculation part |
| 643: | Calibration coefficient calculation part |
| 644: | Moving distance acquisition part |
| 645: | Calibration coefficient acquisition part |
| 646: | Calibration processing part |
| 781: | Data transmission part |
| 782: | Data reception part |
| 783: | Vector synthesis processing part |

1-9. (canceled)

10. A moving distance calculation method comprising:
acquiring, via processing circuitry, an acceleration from an acceleration sensor during a plurality of reciprocations;
performing, via the processing circuitry, gravity correction processing for the acceleration using a gravity correction value corresponding to a gravitational acceleration;
calculating, via the processing circuitry, a velocity by performing integration processing for the acceleration that has been subjected to the gravity correction processing;
calculating, via the processing circuitry, a distance by performing integration processing for the velocity; and
extracting from the distance, via the processing circuitry, feature points corresponding to a start point and an end point in a forward stroke of a reciprocation and calculating a moving distance of the forward stroke of the reciprocation based on a difference value of the distance between the extracted start and end points.

11. The moving distance calculation method according to claim 10, wherein
the gravity correction value is a prescribed value or a value obtained based on the gravitational acceleration in a stationary state which is acquired from the acceleration sensor in the stationary state before a start of the reciprocation.

12. The moving distance calculation method according to claim 10, further comprising:
extracting two feature points from feature points repeatedly appearing in waveforms of the velocity or the distance obtained in a plurality of past reciprocations as a start feature point and an end feature point;
calculating a tilt correction coefficient based on a tilt of a virtual straight line extending from the start feature point to the end feature point in the waveform of the velocity or the distance; and
correcting the velocity or the distance using a tilt correction value obtained based on an elapsed time from the start feature point and the tilt correction coefficient.

13. The moving distance calculation method according to claim 12, comprising:
extracting two feature points from feature points repeatedly appearing in the velocity obtained in the plurality of the past reciprocations as a velocity start feature point and a velocity end feature point;
calculating a velocity tilt correction coefficient based on the tilt of the virtual straight line extending from the velocity start feature point to the velocity end feature point in the waveform of the velocity;
correcting the velocity using a velocity tilt correction value obtained based on an elapsed time from the velocity start feature point and the velocity tilt correction coefficient;
extracting two feature points from feature points repeatedly appearing in the waveform of the distance obtained in the plurality of the past reciprocations as a distance start feature point and a distance end feature point;
calculating a distance tilt correction coefficient based on the tilt of the virtual straight line extending from the distance start feature point to the distance end feature point in the waveform of the distance; and
correcting the distance using a distance tilt correction value obtained based on an elapsed time from the distance start feature point and the distance tilt correction coefficient.

14. The moving distance calculation method according to claim 10, further comprising correcting the velocity to bring a center of a change in the velocity to 0.

15. The moving distance calculation method according to claim 14, wherein the center is an intermediate value between a local maximum value and a local minimum value in one or more reciprocations.

16. The moving distance calculation method according to claim 10, further comprising:
acquiring a preliminary acceleration from the acceleration sensor in a stationary state before a start of the reciprocation;
calculating a preliminary velocity by performing integration processing for the preliminary acceleration;
calculating a preliminary velocity coefficient representing a degree of change in the preliminary velocity over time;
calculating a preliminary velocity correction value based on an elapsed time from the start of the reciprocation and the preliminary velocity coefficient; and
correcting the velocity using the preliminary velocity correction value, wherein the distance is calculated using integration processing for the corrected velocity.

17. The moving distance calculation method according to claim 10, further comprising:
acquiring a calibration acceleration from the acceleration sensor when the plurality of reciprocations are performed at a prescribed frequency by a prescribed moving distance;
performing, based on the calibration acceleration, calibration gravity correction processing for the calibration acceleration using the gravity correction value;
calculating a calibration velocity by performing integration processing for the calibration acceleration that has been subjected to the calibration gravity correction processing;
calculating a calibration distance by performing integration processing for the calibration velocity;
extracting, from the calibration distance, feature points corresponding to a start point and an end point of the forward stroke of the reciprocation and calculating, as a calibration moving distance, a moving distance of the forward stroke of the reciprocation based on a difference value of the calibration distance between the extracted start and end points;
calculating a calibration coefficient based on a ratio of the calibration moving distance relative to the prescribed moving distance; and
calibrating the moving distance using the calibration coefficient.

18. A moving distance calculation method used in an information processing system having a first information processing device put on an arm of a user and a second information processing device communicably connected to the first information processing device, the method comprising:
acquiring an acceleration from an acceleration sensor during a plurality of reciprocations of the first information processing device;
performing gravity correction processing for the acceleration using a gravity correction value corresponding to a gravitational acceleration;
calculating a velocity by performing integration processing for the acceleration that has been subjected to the gravity correction processing;
calculating a distance by performing integration processing for the velocity;
extracting, from the distance, feature points corresponding to a start point and an end point in a forward stroke of a reciprocation and calculating a moving distance of the forward stroke of the reciprocation based on a difference value of the distance between the extracted start and end points; and
transmitting the acceleration, the velocity, and the distance or the moving distance from the first information processing device to the second information processing device.

19. An information processing device comprising processing circuitry configured to:
acquire an acceleration from an acceleration sensor during a plurality of reciprocations of the information processing device,
perform gravity correction processing for the acceleration using a gravity correction value corresponding to a gravitational acceleration,
calculate a velocity by performing integration processing for the acceleration that has been subjected to the gravity correction processing,
calculate a distance by performing integration processing for the velocity, and
extract, from the distance, feature points corresponding to a start point and an end point in a forward stroke of a reciprocation and calculate a moving distance of the forward stroke of the reciprocation based on a difference value of the distance between the extracted start and end points.

20. An information processing device comprising processing circuitry configured to:
acquire an acceleration from an acceleration sensor during a plurality of reciprocations of the information processing device,
perform gravity correction processing for the acceleration using a gravity correction value corresponding to a gravitational acceleration,
calculate a velocity by performing integration processing for the acceleration that has been subjected to the gravity correction processing,
calculate a distance by performing integration processing for the velocity,
extract, from the distance, feature points corresponding to a start point and an end point in a forward stroke of a reciprocation and calculate a moving distance of the forward stroke of the reciprocation based on a difference value of the distance between the extracted start and end points, and
transmit the acceleration, the velocity, and the distance or the moving distance to a second information processing device, the second information processing device communicably connected to the information processing device.

21. A non-transitory computer-readable storage medium for storing computer-readable instructions that, when executed by a computer, cause the computer to perform a moving distance calculation method, the method comprising:
acquiring an acceleration from an acceleration sensor during a plurality of reciprocations;
performing gravity correction processing for the acceleration using a gravity correction value corresponding to a gravitational acceleration;
calculating a velocity by performing integration processing for the acceleration that has been subjected to the gravity correction processing;
calculating a distance by performing integration processing for the velocity; and
extracting, from the distance, feature points corresponding to a start point and an end point in a forward stroke of a reciprocation and calculating a moving distance of the forward stroke of the reciprocation based on a difference value of the distance between the extracted start and end points.

22. The non-transitory computer-readable storage medium according to claim 21, wherein the gravity correction value is a prescribed value or a value obtained based on the gravitational acceleration in a stationary state which is acquired from the acceleration sensor in the stationary state before a start of the reciprocation.

23. The non-transitory computer-readable storage medium according to claim 21, the method further comprising:
extracting two feature points from feature points repeatedly appearing in waveforms of the velocity or the distance obtained in a plurality of past reciprocations as a start feature point and an end feature point;
calculating a tilt correction coefficient based on a tilt of a virtual straight line extending from the start feature point to the end feature point in the waveform of the velocity or the distance; and
correcting the velocity or the distance using a tilt correction value obtained based on an elapsed time from the start feature point and the tilt correction coefficient.

24. The non-transitory computer-readable storage medium according to claim 23, the method comprising:
extracting two velocity feature points from feature points repeatedly appearing in the velocity obtained in the plurality of the past reciprocations as a velocity start feature point and a velocity end feature point;
calculating a velocity tilt correction coefficient based on the tilt of the virtual straight line extending from the velocity start feature point to the velocity end feature point in the waveform of the velocity;
correcting the velocity using a velocity tilt correction value obtained based on an elapsed time from the velocity start feature point and the velocity tilt correction coefficient;
extracting two distance feature points from feature points repeatedly appearing in the waveform of the distance obtained in the plurality of the past reciprocations as a distance start feature point and a distance end feature point;
calculating a distance tilt correction coefficient based on the tilt of the virtual straight line extending from the distance start feature point to the distance end feature point in the waveform of the distance; and
correcting the distance using a distance tilt correction value obtained based on an elapsed time from the distance start feature point and the distance tilt correction coefficient.

25. The non-transitory computer-readable storage medium according to claim 21, the method further comprising correcting the velocity so as to bring a center of a change in the velocity to 0.

26. The non-transitory computer-readable storage medium according to claim 25, wherein the center is an intermediate value between a local maximum value and a local minimum value in one or more reciprocations.

27. The non-transitory computer-readable storage medium according to claim 21, the method further comprising:
acquiring a preliminary acceleration from the acceleration sensor in a stationary state before a start of the reciprocation;
calculating a preliminary velocity by performing integration processing of the preliminary acceleration;
calculating a preliminary velocity coefficient representing a degree of change in the preliminary velocity over time;
calculating a preliminary velocity correction value based on an elapsed time from the start of the reciprocation and the preliminary velocity coefficient; and
correcting the velocity using the preliminary velocity correction value, wherein
the distance is calculated using integration processing for the corrected velocity.

28. The non-transitory computer-readable storage medium according to claim 21, the method comprising:
acquiring a calibration acceleration from the acceleration sensor when the plurality of reciprocations are performed at a prescribed frequency by a prescribed moving distance;
performing, based on the calibration acceleration, calibration gravity correction processing for the calibration acceleration using the gravity correction value;
calculating a calibration velocity by performing integration processing for the calibration acceleration that has been subjected to the calibration gravity correction processing;
calculating a calibration distance by performing integration processing for the calibration velocity;
extracting, from the calibration distance, feature points corresponding to a start point and an end point of the forward stroke of the reciprocation and calculating, as a calibration moving distance, a moving distance of the forward stroke of the reciprocation based on a difference value of the calibration distance between the extracted start and end points;
calculating a calibration coefficient based on a ratio of the calibration moving distance relative to the prescribed moving distance; and
calibrating the moving distance using the calibration coefficient.

29. The non-transitory computer-readable storage medium according to claim 21, the method further comprising transmitting the acceleration, the velocity, and the distance or the moving distance to an information processing device.
